SEQUENCE LISTING (21 sequences)

SEQ ID NO: 1 - DNA, 3600 bp, hybrid
atggagacagacacactcctgctatgggtactgctgctctgggttccaggttccactggtgacgaagatcaggtagatccgcggttaatcgacggtaagcttagccgagatcaataccatgttttgtttgattcctatagagacaatattgctggaaagtcctttcagaatcggctttgtctgcccatgccgattgacgttgtttacacctgggtgaatggcacagatcttgaactactgaaggaactacagcaggtcagagaacagatggaggaggagcagaaagcaatgagagaaatccttgggaaaaacacaacggaacctactaagaagagtgagaagcagttagagtgtttgctaacacactgcattaaggtgccaatgcttgtcctggacccagccctgccagccaacatcaccctgaaggacctgccatctctttatccttcttttcattctgccagtgacattttcaatgttgcaaaaccaaaaaacccttctaccaatgtctcagttgttgtttttgacagtactaaggatgttgaagatgcccactctggactgcttaaaggaaatagcagacagacagtatggaggggctacttgacaacagataaagaagtccctggattagtgctaatgcaagatttggctttcctgagtggatttccaccaacattcaaggaaacaaatcaactaaaaacaaaattgccagaaaatctttcctctaaagtcaaactgttgcagttgtattcagaggccagtgtagcgcttctaaaactgaataaccccaaggattttcaagaattgaataagcaaactaagaagaacatgaccattgatggaaaagaactgaccataagtcctgcatatttattatgggatctgagcgccatcagccagtctaagcaggatgaagacatctctgccagtcgttttgaagataacgaagaactgaggtactcattgcgatctatcgagaggcatgcaccatgggttcggaatattttcattgtcaccaacgggcagattccatcctggctgaaccttgacaatcctcgagtgacaatagtaacacaccaggatgtttttcgaaatttgagccacttgcctacctttagttcacctgctattgaaagtcacgttcatcgcatcgaagggctgtcccagaagtttatttacctaaatgatgatgtcatgtttgggaaggatgtctggccagatgatttttacagtcactccaaaggccagaaggtttatttgacatggcctgtgccaaactgtgccgagggctgcccaggttcctggattaaggatggctattgtgacaaggcttgtaataattcagcctgcgattgggatggtggggattgctctggaaacagtggagggagtcgctatattgcaggaggtggaggtactgggagtattggagttggacagccctggcagtttggtggaggaataaacagtgtctcttactgtaatcagggatgtgcgaattcctggctcgctgataagttctgtgaccaagcatgcaatgtcttgtcctgtgggtttgatgctggcgactgtgggcaagatcattttcatgaattgtataaagtgatccttctcccaaaccagactcactatattattccaaaaggtgaatgcctgccttatttcagctttgcagaagtagccaaaagaggagttgaaggtgcctatagtgacaatccaataattcgacatgcttctattgccaacaagtggaaaaccatccacctcataatgcacagtggaatgaatgccaccacaatacattttaatctcacgtttcaaaatacaaacgatgaagagttcaaaatgcagataacagtggaggtggacacaagggagggaccaaaactgaattctacggcccagaagggttacgaaaatttagttagtcccataacacttcttccagaggcggaaatcctttttgaggatattcccaaagaaaaacgcttcccgaagtttaagagacatgatgttaactcaacaaggagagcccaggaagaggtgaaaattcccctggtaaatatttcactccttccaaaagacgcccagttgagtctcaataccttggatttgcaactggaacatggagacatcactttgaaaggatacaatttgtccaagtcagccttgctgagatcatttctgatgaactcacagcatgctaaaataaaaaatcaagctataataacagatgaaacaaatgacagtttggtggctccacaggaaaaacaggttcataaaagcatcttgccaaacagcttaggagtgtctgaaagattgcagaggttgacttttcctgcagtgagtgtaaaagtgaatggtcatgaccagggtcagaatccacccctggacttggagaccacagcaagatttagagtggaaactcacacccaaaaaaccataggcggaaatgtgacaaaagaaaagcccccatctctgattgttccactggaaagccagatgacaaaagaaaagaaaatcacagggaaagaaaaagagaacagtagaatggaggaaaatgctgaaaatcacataggcgttactgaagtgttacttggaagaaagctgcagcattacacagatagttacttgggctttttgccatgggagaaaaaaaagtatttcctagatcttctcgacgaagaagagtcattgaagacacaattggcctacttcactgatagcaagaatagagccagatacaagagagatacatttgcagattccctcagatatgtaaataaaattctaaatagcaagtttggattcacatcgcggaaagtccctgctcacatgcctcacatgattgaccggattgttatgcaagaactgcaagatatgttccctgaagaatttgacaagacgtcatttcacaaagtgcgccattctgaggatatgcagtttgccttctcttatttttattatctcatgagtgcagtgcagccactgaatatatctcaagtctttgatgaagttgatacagatcaatctggtgtcttgtctgacagagaaatccgaacactggctaccagaattcacgaactgccgttaagtttgcaggatttgacaggtctggaacacatgctaataaattgctcaaaaatgcttcctgctgatatcacgcagctaaataatattccaccaactcaggaatcctactatgatcccaacctgccaccggtcactaaaagtctagtaacaaactgtaaaccagtaactgacaaaatccacaaagcatataaggacaaaaacaaatataggtttgaaatcatgggagaagaagaaatcgcttttaaaatgattcgtaccaacgtttctcatgtggttggccagttggatgacataagaaaaaaccctaggaagtttgtttgcctgaatgacaacattgaccacaatcataaagatgctcagacagtgaaggctgttctcagggacttctatgaatccatgttccccataccttcccaatttgaactgccaagagagtatcgaaaccgtttccttcatatgcatgagctgcaggaatggagggcttatcgagacaaattgaagtag

SEQ ID NO: 2 - protein, 1199 aa, hybrid
METDTLLLWVLLLWVPGSTGDEDQVDPRLIDGKLSRDQYHVLFDSYRDNIAGKSFQNRLCLPMPIDVVYTWVNGTDLELLKELQQVREQMEEEQKAMREILGKNTTEPTKKSEKQLECLLTHCIKVPMLVLDPALPANITLKDLPSLYPSFHSASDIFNVAKPKNPSTNVSVVVFDSTKDVEDAHSGLLKGNSRQTVWRGYLTTDKEVPGLVLMQDLAFLSGFPPTFKETNQLKTKLPENLSSKVKLLQLYSEASVALLKLNNPKDFQELNKQTKKNMTIDGKELTISPAYLLWDLSAISQSKQDEDISASRFEDNEELRYSLRSIERHAPWVRNIFIVTNGQIPSWLNLDNPRVTIVTHQDVFRNLSHLPTFSSPAIESHVHRIEGLSQKFIYLNDDVMFGKDVWPDDFYSHSKGQKVYLTWPVPNCAEGCPGSWIKDGYCDKACNNSACDWDGGDCSGNSGGSRYIAGGGGTGSIGVGQPWQFGGGINSVSYCNQGCANSWLADKFCDQACNVLSCGFDAGDCGQDHFHELYKVILLPNQTHYIIPKGECLPYFSFAEVAKRGVEGAYSDNPIIRHASIANKWKTIHLIMHSGMNATTIHFNLTFQNTNDEEFKMQITVEVDTREGPKLNSTAQKGYENLVSPITLLPEAEILFEDIPKEKRFPKFKRHDVNSTRRAQEEVKIPLVNISLLPKDAQLSLNTLDLQLEHGDITLKGYNLSKSALLRSFLMNSQHAKIKNQAIITDETNDSLVAPQEKQVHKSILPNSLGVSERLQRLTFPAVSVKVNGHDQGQNPPLDLETTARFRVETHTQKTIGGNVTKEKPPSLIVPLESQMTKEKKITGKEKENSRMEENAENHIGVTEVLLGRKLQHYTDSYLGFLPWEKKKYFLDLLDEEESLKTQLAYFTDSKNRARYKRDTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLK

SEQ ID NO: 3 - DNA, 5597 bp, Homo sapiens
cggagccgagcgggcgtccgtcgccggagctgcaatgagcggcgcccggaggctgtgacctgcgcgcggcggcccgaccggggcccctgaatggcggctcgctgaggcggcggcggcggcggcggctcaggctcctcggggcgtggcgtggcggtgaaggggtgatgctgttcaagctcctgcagagacaaacctatacctgcctgtcccacaggtatgggctctacgtgtgcttcttgggcgtcgttgtcaccatcgtctccgccttccagttcggagaggtggttctggaatggagccgagatcaataccatgttttgtttgattcctatagagacaatattgctggaaagtcctttcagaatcggctttgtctgcccatgccgattgacgttgtttacacctgggtgaatggcacagatcttgaactactgaaggaactacagcaggtcagagaacagatggaggaggagcagaaagcaatgagagaaatccttgggaaaaacacaacggaacctactaagaagagtgagaagcagttagagtgtttgctaacacactgcattaaggtgccaatgcttgtactggacccagccctgccagccaacatcaccctgaaggacgtgccatctctttatccttcttttcattctgccagtgacattttcaatgttgcaaaaccaaaaaacccttctaccaatgtctcagttgttgtttttgacagtactaaggatgttgaagatgcccactctggactgcttaaaggaaatagcagacagacagtatggagggggtacttgacaacagataaagaagtccctggattagtgctaatgcaagatttggctttcctgagtggatttccaccaacattcaaggaaacaaatcaactaaaaacaaaattgccagaaaatctttcctctaaagtcaaactgttgcagttgtattcagaggccagtgtagcgcttctaaaactgaataaccccaaggattttcaagaattgaataagcaaactaagaagaacatgaccattgatggaaaagaactgaccataagtcctgcatatttattatgggatctgagcgccatcagccagtctaagcaggatgaagacatctctgccagtcgttttgaagataacgaagaactgaggtactcattgcgatctatcgagaggcatgcaccatgggttcggaatattttcattgtcaccaacgggcagattccatcctggctgaaccttgacaatcctcgagtgacaatagtaacacaccaggatgtttttcgaaatttgagccacttgcctacctttagttcacctgctattgaaagtcacattcatcgcatcgaagggctgtcccagaagtttatttacctaaatgatgatgtcatgtttgggaaggatgtctggccagatgatttttacagtcactccaaaggccagaaggtttatttgacatggcctgtgccaaactgtgccgagggctgcccaggttcctggattaaggatggctattgtgacaaggcttgtaataattcagcctgcgattgggatggtggggattgctctggaaacagtggagggagtcgctatattgcaggaggtggaggtactgggagtattggagttggacacccctggcagtttggtggaggaataaacagtgtctcttactgtaatcagggatgtgcgaattcctggctcgctgataagttctgtgaccaagcatgcaatgtcttgtcctgtgggtttgatgctggcgactgtgggcaagatcattttcatgaattgtataaagtgatccttctcccaaaccagactcactatattattccaaaaggtgaatgcctgccttatttcagctttgcagaagtagccaaaagaggagttgaaggtgcctatagtgacaatccaataattcgacatgcttctattgccaacaagtggaaaaccatccacctcataatgcacagtggaatgaatgccaccacaatacattttaatctcacgtttcaaaatacaaacgatgaagagttcaaaatgcagataacagtggaggtggacacaagggagggaccaaaactgaattctacggcccagaagggttacgaaaatttagttagtcccataacacttcttccagaggcggaaatcctttttgaggatattcccaaagaaaaacgcttcccgaagtttaagagacatgatgttaactcaacaaggagagcccaggaagaggtgaaaattcccctggtaaatatttcactccttccaaaagacgcccagttgagtctcaataccttggatttgcaactggaacatggagacatcactttgaaaggatacaatttgtccaagtcagccttgctgagatcatttctgatgaactcacagcatgctaaaataaaaaatcaagctataataacagatgaaacaaatgacagtttggtggctccacaggaaaaacaggttcataaaagcatcttgccaaacagcttaggagtgtctgaaagattgcagaggttgacttttcctgcagtgagtgtaaaagtgaatggtcatgaccagggtcagaatccacccctggacttggagaccacagcaagatttagagtggaaactcacacccaaaaaaccataggcggaaatgtgacaaaagaaaagcccccatctctgattgttccactggaaagccagatgacaaaagaaaagaaaatcacagggaaagaaaaagagaacagtagaatggaggaaaatgctgaaaatcacataggcgttactgaagtgttacttggaagaaagctgcagcattacacagatagttacttgggctttttgccatgggagaaaaaaaagtatttccaagatcttctcgacgaagaagagtcattgaagacacaattggcatacttcactgatagcaaaaatactgggaggcaactaaaagatacatttgcagattccctcagatatgtaaataaaattctaaatagcaagtttggattcacatcgcggaaagtccctgctcacatgcctcacatgattgaccggattgttatgcaagaactgcaagatatgttccctgaagaatttgacaagacgtcatttcacaaagtgcgccattctgaggatatgcagtttgccttctcttatttttattatctcatgagtgcagtgcagccactgaatatatctcaagtctttgatgaagttgatacagatcaatctggtgtcttgtctgacagagaaatccgaacactggctaccagaattcacgaactgccgttaagtttgcaggatttgacaggtctggaacacatgctaataaattgctcaaaaatgcttcctgctgatatcacgcagctaaataatattccaccaactcaggaatcctactatgatcccaacctgccaccggtcactaaaagtctagtaacaaactgtaaaccagtaactgacaaaatccacaaagcatataaggacaaaaacaaatataggtttgaaatcatgggagaagaagaaatcgcttttaaaatgattcgtaccaacgtttctcatgtggttggccagttggatgacataagaaaaaaccctaggaagtttgtttgcctgaatgacaacattgaccacaatcataaagatgctcagacagtgaaggctgttctcagggacttctatgaatccatgttccccataccttcccaatttgaactgccaagagagtatcgaaaccgtttccttcatatgcatgagctgcaggaatggagggcttatcgagacaaattgaagttttggacccattgtgtactagcaacattgattatgtttactatattctcattttttgctgagcagttaattgcacttaagcggaagatatttcccagaaggaggatacacaaagaagctagtcccaatcgaatcagagtatagaagatcttcatttgaaaaccatctacctcagcatttactgagcattttaaaactcagcttcacagagatgtctttgtgatgtgatgcttagcagtttggcccgaagaaggaaaatatccagtaccatgctgttttgtggcatgaatatagcccactgactaggaattatttaaccaacccactgaaaacttgtgtgtcgagcagctctgaactgattttacttttaaagaatttgctcatggacctgtcatcctttttataaaaaggctcactgacaagagacagctgttaatttcccacagcaatcattgcagactaactttattaggagaagcctatgccagctgggagtgattgctaagaggctccagtctttgcattccaaagccttttgctaaagttttgcactttttttttttcatttcccatttttaagtagttactaagttaactagttattcttgcttctgagtataacgaattgggatgtctaaacctatttttatagatgttatttaaataatgcagcaatatcacctcttattgacaatacctaaattatgagttttattaatatttaagactgtaaatggtcttaaaccactaactactgaagagctcaatgattgacatctgaaatgctttgtaattattgacttcagcccctaagaatgctatgatttcacgtgcaggtctaatttcaacaggctagagttagtactacttaccagatgtaattatgttttggaaatgtacatattcaaacagaagtgcctcattttagaaatgagtagtgctgatggcactggcacattacagtggtgtcttgtttaatactcattggtatattccagtagctatctctctcagttggtttttgatagaacagaggccagcaaactttctttgtaaaaggctggttagtaaattattgcaggccacctgtgtctttgtcatacattcttcttgctgttgtttagtttgttttttttcaaacaaccctctaaaaatgtaaaaaccatgtttagcttgcagctgtacaaaaactgcccaccagccagatgtgaccctcaggccatcatttgccaatcactgagaattatttttgttgttgttgttgttgttgtttttgagacagagtctctctctgttgcccaggctggagtgcagtggcgcaatctcagctcactgcaacctccgcctcccgggttcaagcagttctgtctcagccttctgagtagctgggactacaggtgcatgccaccacaccctgctaatttttgtatttttagtagagacgggggttccaccatattggtcaggcttatcttgaactcctgacctcaggtgatccacctgcctctgcctcccaaagtgctgagattacaggcataagccagtgcacccagccgagaattagtatttttatgtatggttaaaccttggcgtctagccatattttatgtcataatacaatggatttgtgaagagcagattccatgagtaactctgacaggtattttagatcatgatctcaacaatattcctcccaaatggcatacatcttttgtacaaagaacttgaaatgtaaatactgtgtttgtgctgtaagagttgtgtatttcaaaaactgaaatctcataaaaagttaaattttgaaaa

SEQ ID NO: 4 - protein, 928 aa, Homo sapiens
MLFKLLQRQTYTCLSHRYGLYVCFLGVVVTIVSAFQFGEVVLEWSRDQYHVLFDSYRDNIAGKSFQNRLCLPMPIDVVYTWVNGTDLELLKELQQVREQMEEEQKAMREILGKNTTEPTKKSEKQLECLLTHCIKVPMLVLDPALPANITLKDVPSLYPSFHSASDIFNVAKPKNPSTNVSVVVFDSTKDVEDAHSGLLKGNSRQTVWRGYLTTDKEVPGLVLMQDLAFLSGFPPTFKETNQLKTKLPENLSSKVKLLQLYSEASVALLKLNNPKDFQELNKQTKKNMTIDGKELTISPAYLLWDLSAISQSKQDEDISASRFEDNEELRYSLRSIERHAPWVRNIFIVTNGQIPSWLNLDNPRVTIVTHQDVFRNLSHLPTFSSPAIESHIHRIEGLSQKFIYLNDDVMFGKDVWPDDFYSHSKGQKVYLTWPVPNCAEGCPGSWIKDGYCDKACNNSACDWDGGDCSGNSGGSRYIAGGGGTGSIGVGHPWQFGGGINSVSYCNQGCANSWLADKFCDQACNVLSCGFDAGDCGQDHFHELYKVILLPNQTHYIIPKGECLPYFSFAEVAKRGVEGAYSDNPIIRHASIANKWKTIHLIMHSGMNATTIHFNLTFQNTNDEEFKMQITVEVDTREGPKLNSTAQKGYENLVSPITLLPEAEILFEDIPKEKRFPKFKRHDVNSTRRAQEEVKIPLVNISLLPKDAQLSLNTLDLQLEHGDITLKGYNLSKSALLRSFLMNSQHAKIKNQAIITDETNDSLVAPQEKQVHKSILPNSLGVSERLQRLTFPAVSVKVNGHDQGQNPPLDLETTARFRVETHTQKTIGGNVTKEKPPSLIVPLESQMTKEKKITGKEKENSRMEENAENHIGVTEVLLGRKLQHYTDSYLGFLPWEKKKYFQDLLDEEESLKTQLAYFTDSKNTGRQLK

SEQ ID NO: 5 - protein, 328 aa, Homo sapiens
DTFADSLRYVNKILNSKFGFTSRKVPAHMPHMIDRIVMQELQDMFPEEFDKTSFHKVRHSEDMQFAFSYFYYLMSAVQPLNISQVFDEVDTDQSGVLSDREIRTLATRIHELPLSLQDLTGLEHMLINCSKMLPADITQLNNIPPTQESYYDPNLPPVTKSLVTNCKPVTDKIHKAYKDKNKYRFEIMGEEEIAFKMIRTNVSHVVGQLDDIRKNPRKFVCLNDNIDHNHKDAQTVKAVLRDFYESMFPIPSQFELPREYRNRFLHMHELQEWRAYRDKLKFWTHCVLATLIMFTIFSFFAEQLIALKRKIFPRRRIHKEASPNRIRV

SEQ ID NO: 6 - DNA, 1219 bp, Homo sapiens
gtagagcgcaggtgcgcggctcgatggcggcggggctggcgcggctcctgttgctcctcgggctctcggccggcgggcccgcgccggcaggtgcagcgaagatgaaggtggtggaggagcccaacgcgtttggggtgaacaacccgttcttgcctcaggccagtcgcctccaggccaagagggatccttcacccgtgtctggacccgtgcatctcttccgactctcgggcaagtgcttcagcctggtggagtccacgtacaagtatgagttctgcccgttccacaacgtgacccagcacgagcagaccttccgctggaacgcctacagtgggatcctcggcatctggcacgagtgggagatcgccaacaacaccttcacgggcatgtggatgagggacggtgacgcctgccgttcccggagccggcagagcaaggtggagctggcgtgtggaaaaagcaaccggctggcccatgtgtccgagccgagcacctgcgtctatgcgctgacgttcgagacccccctcgtctgccacccccacgccttgctagtgtacccaaccctgccagaggccctgcagcggcagtgggaccaggtagagcaggacctggccgatgagctgatcaccccccagggccatgagaagttgctgaggacactttttgaggatgctggctacttaaagaccccagaagaaaatgaacccacccagctggagggaggtcctgacagcttggggtttgagaccctggaaaactgcaggaaggctcataaagaactctcaaaggagatcaaaaggctgaaaggtttgctcacccagcacggcatcccctacacgaggcccacagaaacttccaacttggagcacttgggccacgagacgcccagagccaagtctccagagcagctgcggggtgacccaggactgcgtgggagtttgtgaccttgtggtgggagagcagaggtggacgcggccgagagccctacagagaagctggctggtaggacccgcaggaccagctgaccaggcttgtgctcagagaagcagacaaaacaaagattcaaggttttaattaattcccatactgataaaaataactccatgaattctgtaaaccattgcataaatgctatagtgtaaaaaaatttaaacaagtgttaactttaaacagttcgctacaagtaaatgattataaatactaaaaaaaaaaaaaaaaaaaa

SEQ ID NO: 7 - protein, 305 aa, Homo sapiens
MAAGLARLLLLLGLSAGGPAPAGAAKMKVVEEPNAFGVNNPFLPQASRLQAKRDPSPVSGPVHLFRLSGKCFSLVESTYKYEFCPFHNVTQHEQTFRWNAYSGILGIWHEWEIANNTFTGMWMRDGDACRSRSRQSKVELACGKSNRLAHVSEPSTCVYALTFETPLVCHPHALLVYPTLPEALQRQWDQVEQDLADELITPQGHEKLLRTLFEDAGYLKTPEENEPTQLEGGPDSLGFETLENCRKAHKELSKEIKRLKGLLTQHGIPYTRPTETSNLEHLGHETPRAKSPEQLRGDPGLRGSL

SEQ ID NO: 8 - DNA, 5229 bp, Mus musculus
ggcggtgaaggggtgatgctgttcaagctcctgcagagacagacctatacctgcctatcccacaggtatgggctctacgtctgcttcgtgggcgtcgttgtcaccatcgtctcggctttccagttcggagaggtggttctggaatggagccgagatcagtaccatgttttgtttgattcctacagagacaacattgctgggaaatcctttcagaatcggctctgtctgcccatgccaatcgacgtggtttacacctgggtgaatggcactgaccttgaactgctaaaggagctacagcaggtccgagagcacatggaggaagagcagagagccatgcgggaaaccctcgggaagaacacaaccgaaccgacaaagaagagtgagaagcagctggaatgtctgctgacgcactgcattaaggtgcccatgcttgttctggacccggccctgccagccaccatcaccctgaaggatctgccaaccctttacccatctttccacgcgtccagcgacatgttcaatgttgcgaaaccaaaaaatccgtctacaaatgtccccgttgtcgtttttgacactactaaggatgttgaagacgcccatgctggaccgtttaagggaggccagcaaacagatgtttggagagcctacttgacaacagacaaagacgcccctggcttagtgctgatacaaggcttggcgttcctgagtggattcccaccgaccttcaaggagacgagtcaactgaagacaaagctgccaagaaaagctttccctctaaaaataaagctgttgcggctgtactcggaggccagtgtcgctcttctgaaattgaataatcccaagggtttccaagagctgaacaagcagaccaagaagaacatgaccatcgatgggaaggaactgaccatcagccctgcgtatctgctgtgggacctgagtgccatcagccagtccaagcaggatgaggacgcgtctgccagccgctttgaggataatgaagagctgaggtactcgctgcgatctatcgagagacacgcgccatgggtacggaatattttcattgtcaccaacgggcagattccatcctggctgaaccttgacaaccctcgagtgaccatagtgacccaccaggacattttccaaaatctgagccacttgcctactttcagttcccctgctattgaaagtcacattcaccgcatcgaagggctgtcccagaagtttatttatctaaatgacgatgtcatgttcggtaaggacgtctggccggacgatttttacagccactccaaaggtcaaaaggtttatttgacatggcctgtgccaaactgtgcagagggctgcccgggctcctggataaaggacggctattgtgataaggcctgtaatacctcaccctgtgactgggatggcggaaactgctctggtaatactgcagggaaccggtttgttgcaagaggtgggggtaccgggaatattggagctggacagcactggcagtttggtggaggaataaacaccatctcttactgtaaccaaggatgtgcaaactcctggctggctgacaagttctgtgaccaagcctgtaacgtcttatcctgcgggtttgatgctggtgactgtggacaagatcattttcatgaattgtataaagtaacacttctcccaaaccagactcactatgttgtccccaaaggtgaatacctgtcttatttcagctttgcaaacatagccagaaaaagaattgaagggacctacagcgacaaccccatcatccgccacgcgtccattgcaaacaagtggaaaaccctacacctgataatgcccggggggatgaacgccaccacgatctattttaacctcactcttcaaaacgccaacgacgaagagttcaagatccagatagcagtagaggtggacacgagggaggcgcccaaactgaattctacaacccagaaggcctatgaaagtttggttagcccagtgacacctcttcctcaggctgacgtcccttttgaagatgtccccaaagagaaacgcttccccaagatcaggagacatgatgtaaatgcaacagggagattccaagaggaggtgaaaatcccccgggtaaatatttcactccttcccaaagaggcccaggtgaggctgagcaacttggatttgcaactagaacgtggagacatcactctgaaaggatataacttgtccaagtcagccctgctaaggtctttcctggggaattcactagatactaaaataaaacctcaagctaggaccgatgaaacaaaaggcaacctggaggtcccacaggaaaacccttctcacagacgtccacatggctttgctggtgaacacagatcagagagatggactgccccagcagagacagtgaccgtgaaaggccgtgaccacgctttgaatccacccccggtgttggagaccaatgcaagattggcccagcctacactaggcgtgactgtgtccaaagagaacctttcaccgctgatcgttcccccagaaagccacttgccaaaagaagaggagagtgacagggcagaaggcaatgctgtacctgtaaaggagttagtgcctggcagacggttgcagcagaattatccaggctttttgccctgggagaaaaaaaagtatttccaagaccttcttgatgaggaagagtcattgaagacccagttggcgtactttacagaccgcaaacataccgggaggcaactaaaagatacatttgcagactccctccgatacgtcaataaaattctcaacagcaagtttggattcacatccaggaaagtccctgcacacatgccgcacatgattgacaggatcgttatgcaagaactccaagatatgttccctgaagaatttgacaagacttcatttcacaaggtgcgtcactctgaggacatgcagtttgccttctcctacttttattacctcatgagtgcagttcagcccctcaatatttcccaagtctttcatgaagtagacacagaccaatctggtgtcttgtctgatagggaaatccgaacwctggccacgagaattcacgacctacctttaagcttgcaggatttgacaggtttggaacacatgttaataaattgctcaaaaatgctccccgctaatatcactcaactcaacaacatcccaccgactcaggaagcatactacgaccccaacctgcctccggtcactaagagtcttgtcaccaactgtaagccagtaactgacaagatccacaaagcctataaagacaagaacaaatacaggtttgaaatcatgggagaggaagaaatcgctttcaagatgatacgaaccaatgtttctcatgtggttggtcagttggatgacatcagaaaaaaccccaggaagttcgtttgtctgaatgacaacattgaccacaaccataaagatgcccggacagtgaaggctgtcctcagggacttctatgagtccatgtttcccataccttcccagtttgagctgccaagagagtatcggaaccgctttctgcacatgcatgagctccaagaatggcgggcatatcgagacaagctgaagttttggacccactgcgtactagcaacgttgattatatttactatattctcattttttgctgaacagataattgctctgaagcgaaagatatttcccaggaggaggatacacaaagaagctagtccagaccgaatcagggtgtagaagatcttcatttgaaagtcacctaccttagcatctgtgaacatctccctcctcgacaccacagcggagtccctgtgatgtggcacagaggcagcctcgtggggagaagggacatcgtgcagaccgggttcttctgcaatgggaagagagcccactgacctggaattattcagcacactaagaacctgtgtcaatagcttgtacagcttgtacttttaaaggatttgccgaaggacctgtcggcttgttgacaaaccctccctgacaagctgctggtttcttcccccagttactgcagactgagaaaccagtccatcttgaaagcaagtgcggaggggccccagtctttgcattccaaagctttccagcataatttctggcttgtctcctcctttgatccatttcccatttttttttaaaaaacaataagtggctactaagttagtcattctcacttctcaaaataacaaatcaggatgtcaaaacatttgtatagatcttatttaaataatatagaacgattacttctttagcctatctaaattattgatttttattaacagtcaagtggtcttgaaccgctaacaactactgaagagctcgagattgacgttgaaagtgctttgagcttgtttaactcattccccaagaatactgtgacctcgtgtgcgggcctgattgcgaagggctagtgtcacgtagcagtgctgctcaccggatgtaattatgtcgtggaaatgtacatacagacaaaagtgcctcacttcagaaatgagtagtgctgatggcaccagcgagtgatggtgtccatttggaaacccatgataccttccaatgcccaccctgcttactttatacagagcaggggttaaccaacttctgtcaaagaacagtaaagaacttgagatacatccatctttgtcaaatagttttccttgctaacatttattattgttggtgttttgggaggtttattttattttattgctttgttatttttcaagacggggattctctgtgtagctctggctgtttggtaattcactctaaagaccaggctggccttgaacttagagattcacctgcttctgcttcctgaatggtaggacatgtgcccacattgcctacccaccccccttttggggggggtgagcaactcaataaaaagatgaaaacctgctttagtttgcagctatacaaaagcagcaggcctcagccagacttgacccccggggccattgttggcccacgggagaatcatttttgacgtgggtaagcaaaccctgatattggtcatgctgtgttatgtcattatgtggtggttttgaattttggaagatattttcagtcatgatttcagtagtattcctccaaaatggcacacatttttgtaataagaacttgaaatgtaaatattgtgtttgtgctgtaaattttgtgtatttcaaaaactgaagtttcataaaaaaacacacttattggaaaaaaaaaaaaaaaaaaaaa

SEQ ID NO: 9 - protein, 908 aa, Mus musculus
MLFKLLQRQTYTCLSHRYGLYVCFVGVVVTIVSAFQFGEVVLEWSRDQYHVLFDSYRDNIAGKSFQNRLCLPMPIDVVYTWVNGTDLELLKELQQVREHMEEEQRAMRETLGKNTTEPTKKSEKQLECLLTHCIKVPMLVLDPALPATITLKDLPTLYPSFHASSDMFNVAKPKNPSTNVPVVVFDTTKDVEDAHAGPFKGGQQTDVWRAYLTTDKDAPGLVLIQGLAFLSGFPPTFKETSQLKTKLPRKAFPLKIKLLRLYSEASVALLKLNNPKGFQELNKQTKKNMTIDGKELTISPAYLLWDLSAISQSKQDEDASASRFEDNEELRYSLRSIERHAPWVRNIFIVTNGQIPSWLNLDNPRVTIVTHQDIFQNLSHLPTFSSPAIESHIHRIEGLSQKFIYLNDDVMFGKDVWPDDFYSHSKGQKVYLTWPVPNCAEGCPGSWIKDGYCDKACNTSPCDWDGGNCSGNTAGNRFVARGGGTGNIGAGQHWQFGGGINTISYCNQGCANSWLADKFCDQACNVLSCGFDAGDCGQDHFHELYKVTLLPNQTHYVVPKGEYLSYFSFANIARKRIEGTYSDNPIIRHASIANKWKTLHLIMPGGMNATTIYFNLTLQNANDEEFKIQIAVEVDTREAPKLNSTTQKAYESLVSPVTPLPQADVPFEDVPKEKRFPKIRRHDVNATGRFQEEVKIPRVNISLLPKEAQVRLSNLDLQLERGDITLKGYNLSKSALLRSFLGNSLDTKIKPQARTDETKGNLEVPQENPSHRRPHGFAGEHRSERWTAPAETVTVKGRDHALNPPPVLETNARLAQPTLGVTVSKENLSPLIVPPESHLPKEEESDRAEGNAVPVKELVPGRRLQQNYPGFLPWEKKKYFQDLLDEEESLKTQLAYFTDRKHTGRQLK

SEQ ID NO: 10 - protein, 328 aa, Mus musculus
DTFADSLRYVNKILNSKFGFTSRKVPAHMPHMIDRIVMQELQDMFPEEFDKTSFHKVRHSEDMQFAFSYFYYLMSAVQPLNISQVFHEVDTDQSGVLSDREIRTLATRIHDLPLSLQDLTGLEHMLINCSKMLPANITQLNNIPPTQEAYYDPNLPPVTKSLVTNCKPVTDKIHKAYKDKNKYRFEIMGEEEIAFKMIRTNVSHVVGQLDDIRKNPRKFVCLNDNIDHNHKDARTVKAVLRDFYESMFPIPSQFELPREYRNRFLHMHELQEWRAYRDKLKFWTHCVLATLIIFTIFSFFAEQIIALKRKIFPRRRIHKEASPDRIRV

SEQ ID NO: 11 - DNA, 2070 bp, Mus musculus
  misc_feature (186)..(186): n is a, t, c, or g
gtgagaccctaggagcaatggccgggcggctggctggcttcctgatgttgctggggctcgcgtcgcaggggcccgcgccggcatgtgccgggaagatgaaggtggtggaggagcctaacacattcgggtgagcggatcacggtcctgcggcttggggaccgagcctggctggttcttctgaccttntcaattccataggctgaataacccgttcttgccccaggcaagccgccttcagcccaagagagagccttcagctgtatcccgcaaattaagagaaattaatttcaaacgatttagaaagtattctagccaggcgatgatggcgcacgcctttaatcccagcacttgggaggcagaggcaggcagatttccgagttcaaggccatcagaactgactgtacatcttagtacagtttagcatgtgatcagagatctgaatcacaaagctgggcctgcgtggtaaagcaggtcctttctaataaggttgcagtttagattttctttcttaactcttttattctttgagacagggtttctcaacagtgggtgtcctggaactcacttttgtaaaccaggctgcccttaaactcacaaagctctgtcagcctctgcctcctgagtgctgggattaaaggtccacaccctgttcattcatttttaatttttgagactgggtctcattatgtggccctagacagatactgagagcctcctccacaggaacaagcatgggaatcctgccacagacaaccagttctgtggtctggagatgagtttgtcagtccctaggagttaggtcagcctgcctctgcattcccaataatttaggaaaggagcttggggcgttctggccttgatggttagtgccctcctgccaaccttagcttccagctttaggggtagcagagtttataccgatgctaaactgctgttgtgttcttccccagggcccctgcatctcttcagacttgctggcaagtgctttagcctagtggagtccacgtgagtgccaggctggtgggtggagtgggcggagtctgcagagctcctgatgtgcctgtgtttcccaggtacaagtatgaattctgccctttccacaacgtcacccagcacgagcagaccttccgctggaatgcctacagcgggatccttggcatctggcatgagtgggaaatcatcaacaataccttcaagggcatgtggatgactgatggggactcctgccactcccggagccggcagagcaaggtggagctcacctgtggaaagatcaaccgactggcccacgtgtctgagccaagcacctgtgtctatgcattgacattcgagacccctcttgtttgccatccccactctttgttagtgtatccaactctgtcagaagccctgcagcagcccttggaccaggtggaacaggacctggcagatgaactgatcacaccacagggctatgagaagttgctaagggtactttttgaggatgctggctacttaaaggtcccaggagaaacccatcccacccagctggcaggaggttccaagggcctggggcttgagactctggacaactgtagaaaggcacatgcagagctgtcacaggaggtacaaagactgacgagtctgctgcaacagcatggaatcccccacactcagcccacaggtcagtctgcctgccctggtcagctgccagccactccggggcctgcagcactggggcagatctttattgctacccattctggcagaaaccactcactctcagcacctgggtcagcagctccccataggtgcaatcgcagcagagcatctgcggagtgacccaggactacgtgggaacatcctgtgagcaaggtggccacgaagaatagaaatatcctgagctttgagtgtcctttcacagagtgaacaaaactggtgtggtgtagacacggcttcttttggcatattctagatcagacagtgtcactgacaaacaagagggacctgctggccagcctttgttgtgcccaaagatccagacaaaataaagattcaaagttttaattaaaaaaaaaaaaaaaggaattc

SEQ ID NO: 12 - protein, 307 aa, Mus musculus
MAGRLAGFLMLLGLASQGPAPACAGKMKVVEEPNTFGLNNPFLPQASRLQPKREPSAVSGPLHLFRLAGKCFSLVESTYKYEFCPFHNVTQHEQTFRWNAYSGILGIWHEWEIINNTFKGMWMTDGDSCHSRSRQSKVELTCGKINRLAHVSEPSTCVYALTFETPLVCHPHSLLVYPTLSEALQQRLDQVEQDLADELITPQGYEKLLRVLFEDAGYLKVPGETHPTQLAGGSKGLGLETLDNCRKAHAELSQEVQRLTSLLQQHGIPHTQPTETTHSQHLGQQLPIGAIAAEHLRSDPGLRGNIL

SEQ ID NO: 13 - DNA, 460 bp, Rattus rattus
attcccaccaacattcaaggagacgagtcagctgaagacaaaactgccagaaaatctttcttctaaaataaaactgttgcagctgtactcggaggccagcgtcgctcttctgaaattgaataaccccaaaggtttccccgagctgaacaagcagaccaagaagaacatgagcatcagtgggaaggaactggccatcagccctgcctatctgctgtgggacctgagcgccatcagccagtccaagcaggatgaagatgtgtctgccagccgcttcgaggataacgaagagctgaggtactcactgagatctatcgagagacatgattccatgagtcctttatgaattctggccatatcttcaatcatgatctcagtagtattcctctgaaatggcacacatttttctaatgagaacttgaaatgtaaatattgtgtttgtgctgtaaattttgtgtatttc

SEQ ID NO: 14 - protein, 113 aa, Rattus rattus
FPPTFKETSQLKTKLPENLSSKIKLLQLYSEASVALLKLNNPKGFPELNKQTKKNMSISGKELAISPAYLLWDLSAISQSKQDEDVSASRFEDNEELRYSLRSIERHDSMSPL

SEQ ID NO: 15 - DNA, 1105 bp, Drosophila melanogaster
  misc_feature (903)..(903): n is a, g, t, or c
ctgcaggaattcggcacgaggcggttcgatgacaagaatgagctgcggtactctctgaggtccctggaaaaacacgccgcatggatcaggcatgtgtacatagtaaccaatggccagattccaagttggctggatctcagctacgaaagggtcacggtggtgccccacgaagtcctggctcccgatcccgaccagctgcccaccttctccagctcggccatcgagacatttctgcaccgcataccaaagctgtccaagaggttcctctacctcaacgacgacatattcctgggagctccgctgtatccggaggacttgtacactgaagcggagggagttcgcgtgtaccaggcatggatggtgcccggctgcgccttggattgcccctggacgtacataggtgatggagcttgcgatcggcactgcaacattgatgcgtgccaatttgatggaggcgactgcagtgaaactgggccagcgagcgatgcccacgtcattccaccaagcaaagaagtgctcgaggtgcagcctgccgctgttccacaatcaagagtccaccgatttcctcagatgggtctccaaaagctgttcaggcgcagctctgccaattttaaggatgttatgcggcaccgcaatgtgtccacactcaaggaactacgtcgcattgtggagcgttttaacaaggccaaactcatgtcgctgaaccccgaactggagacctccagctccgagccacagacaactcagcgccacgggctgcgcaaggaggattttaagtcttccaccgatatttactctcactcgctgattgccaccaatatgttgctgaatagagcctatggctttaaggcacgccatgtcctggcgcacgtgggcttcctaattgacaaggatattgtggangccatgcaacgacgttttaccagcgaattctngacactggccattaacgctttccgagccccaacagatttgcagtacgcattcgcttactacttctttctaatgagcgaaatccaagtnatgagtgtagangaaatcttcgatgaagtcgacaccggacggtttggncacctggtcggatccagaagtgcgaaccntttta

SEQ ID NO: 16 - protein, 502 aa, Drosophila melanogaster
GTRRFDDKNELRYSLRSLEKHAAWIRHVYIVTNGQIPSWLDLSYERVTVVPHEVLAPDPDQLPTFSSSAIETFLHRIPKLSKRFLYLNDDIFLGAPLYPEDLYTEAEGVRVYQAWMVPGCALDCPWTYIGDGACDRHCNIDACQFDGGDCSETGPASDAHVIPPSKEVLEVQPAAVPQSRVHRFPQMGLQKLFRRSSANFKDVMRHRNVSTLKELRRIVERFNKAKLMSLNPELETSSSEPQTTQRHGLRKEDFKSSTDIYSHSLIATNMLLNRAYGFKARHVLAHVGFLIDKDIVEAMQRRFHQQILDTAHQRFRAPTDLQYAFAYYSFLMSETKVMSVEEIFDEFDTDGSATWSDREVRTFLTRIYQPPLDWSAMRYFEEVVQNCTRNLGMHLKVDTVEHSTLVYERYEDSNLPTITRDLVVRCPLLAEALAANFAVRPKYNFHVSPKRTSHSNFMMLTSNLTEVVESLDRLRRNPRKFNCINDNLDANRGEDNEDGAPS

SEQ ID NO: 17 - DNA, 2183 bp, Homo sapiens
atggcgacctccacgggtcgctggcttctcctccggcttgcactattcggcttcctctgggaagcgtccggcggcctcgactcgggggcctcccgcgacgacgacttgctactgccctatccacgcgcgcgcgcgcgcctcccccgggactgcacacgggtgcgcgccggcaaccgcgagcacgagagttggcctccgcctcccgcgactcccggcgccggcggtctggccgtgcgcaccttcgtgtcgcacttcagggaccgcgcggtggccggccacctgacgcgggccgttgagcccctgcgcaccttctcggtgctggagcccggtggacccggcggctgcgcggcgagacgacgcgccaccgtggaggagacggcgcgggcggccgactgccgtgtcgcccagaacggcggcttcttccgcatgaactcgggcgagtgcctggggaacgtggtgagcgacgagcggcgggtgagcagctccggggggctgcagaacgcgcagttcgggatccgccgcgacgggaccctggtcaccgggtacctgtctgaggaggaggtgctggacactgagaacccatttgtgcagctgctgagtggggtcgtgtggctgattcgtaatggaagcatctacatcaacgagagccaagccacagagtgtgacgagacacaggagacaggttcctttagcaaatttgtgaatgtgatatcagccaggacggccattggccacgaccggaaagggcagctggtgctctttcatgcagacggccatacggagcagcgtggcatcaacctgtgggaaatggcggagttcctgctgaaacaggacgtggtcaacgccatcaacctggatgggggtggctctgccacctttgtgctcaacgggaccttggccagttacccgtcagatcactgccaggacaacatgtggcgctgtccccgccaagtgtccaccgtggtgtgtgtgcacgaaccccgctgccagccgcctgactgccacggccacgggacctgcgtggacgggcactgccaatgcaccgggcacttctggcggggtcccggctgtgatgagctggactgtggcccctctaactgcagccagcacggactgtgcacggagaccggctgccgctgtgatgccggatggaccgggtccaactgcagtgaagagtgtccccttggctggcatgggccgggctgccagaggcgttgtaagtgtgagcaccattgtccctgtgaccccaagactggcaactgcagcgtctccagagtaaagcagtgtctccagccacctgaagccaccctgagggcgggagaactctcctttttcaccaggaccgcctggctagccctcaccctggcgctggccttcctcctgctgatcagcattgcagcaaacctgtccttgctcctgtccagagcagagaggaaccggcgcctgcatggggactatgcataccacccgctgcaggagatgaacggggagcctctggccgcagagaaggagcagccagggggcgcccacaaccccttcaaggactgaagcctcaagctgcccggggtggcacgtcgcgaaagcttgtttccccacggtctggcttctgcaggggaaatttcaaggccactggcgtggaccatctgggtgtcctcaatggcccctgtggggcagccaagttcctgatagcacttgtgcctcagcccctcacctggccacctgccagggcacctgcaaccctagcaataccatgctcgctggagaggctcagctgcctgcttctcgcctgcctgtgtctgctgccgagaagcccgtgcccccgggagggctgccgcactgccaaagagtctccctcctcctggggaaggggctgccaacgaaccagactcagtgaccacgtcatgacagaacagcacatcctggccagcacccctggctggagtgggttaaagggacgagtctgccttcctggctgtgacacgggaccccttttctacagacctcatcactggatttgccaactagaattcgatttcctgtcataggaagctccttggaagaagggatggggggatgaaatcatgtttacagacctgttttgtcatcctgctgccaagaagttttttaatcacttgaataaattgatataataaaaggagccaccaggtggtgtgtggattctg

SEQ ID NO: 18 - protein, 515 aa, Homo sapiens
MATSTGRWLLLRLALFGFLWEASGGLDSGASRDDDLLLPYPRARARLPRDCTRVRAGNREHESWPPPPATPGAGGLAVRTFVSHFRDRAVAGHLTRAVEPLRTFSVLEPGGPGGCAARRRATVEETARAADCRVAQNGGFFRMNSGECLGNVVSDERRVSSSGGLQNAQFGIRRDGTLVTGYLSEEEVLDTENPFVQLLSGVVWLIRNGSIYINESQATECDETQETGSFSKFVNVISARTAIGHDRKGQLVLFHADGHTEQRGINLWEMAEFLLKQDVVNAINLDGGGSATFVLNGTLASYPSDHCQDNMWRCPRQVSTVVCVHEPRCQPPDCHGHGTCVDGHCQCTGHFWRGPGCDELDCGPSNCSQHGLCTETGCRCDAGWTGSNCSEECPLGWHGPGCQRRCKCEHHCPCDPKTGNCSVSRVKQCLQPPEATLRAGELSFFTRTAWLALTLALAFLLLISIAANLSLLLSRAERNRRLHGDYAYHPLQEMNGEPLAAEKEQPGGAHNPFKD

SEQ ID NO: 19 - DNA, 2005 bp, Mus musculus
gtttcccgcgacgatgacctgctgctgccttacccactagcgcgcagacgtccctcgcgagactgcgcccgggtgcgctcaggtagcccagagcaggagagctggcctccgccacctctggccacccacgaaccccgggcgccaagccaccacgcggccgtgcgcaccttcgtgtcgcacttcgaggggcgcgcggtggccggccacctgacgcgggtcgccgatcccctacgcactttctcggtgctggagcccggaggagccgggggctgcggcggcagaagcgccgcggctactgtggaggacacagccgtccgggccggttgccgcatcgctcagaacggtggcttcttccgcatgagcactggcgagtgcttggggaacgtggtgagcgacgggcggctggtgagcagctcagggggactgcagaacgcgcagttcggtatccgacgcgatggaaccatagtcaccgggtcctgtcttgaagaagaggttctggatcccgtgaatccgttcgtgcagctgctgagcggagtcgtgtggctcatccgcaatggaaacatctacatcaacgagagccaagccatcgagtgtgacgagacacaggagacaggttcttttagcaaatttgtgaatgtgatgtcagccaggacagccgtgggtcatgaccgtgaggggcagcttatcctcttccatgctgatggacagacggaacagcgtggccttaacctatgggagatggcagagttcctgcgtcaacaagatgtcgtcaatgccatcaacctggatggaggcggttctgctacttttgtgctcaatgggaccctggccagttacccttcagatcactgccaggacaacatgtggcgctgtccccgccaagtgtccactgtggtgtgtgtgcatgaaccgcgctgccagccacccgactgcagtggccatgggacctgtgtggatggccactgtgaatgcaccagccacttctggcggggcgaggcctgcagcgagctggactgtggcccctccaactgcagccagcatgggctgtgcacagctggctgccactgtgatgctgggtggacaggatccaactgcagtgaagagtgtcctctgggctggtatgggccaggttgccagaggccctgccagtgtgagcaccagtgtttctgtgacccgcagactggcaactgcagcatctcccaagtgaggcagtgtctccagccaactgaggctacgccgagggcaggagagctggcctctttcaccaggaccacctggctagccctcaccctgacactaattttcctgctgctgatcagcactggggtcaacgtgtccttgttcctgggctccagggccgagaggaaccggcacctcgacggggactatgtgtatcacccactgcaggaggtgaacggggaagcgctgactgcagagaaggagcacatggaggaaactagcaaccccttcaaggactgaagagctgccccaacggcatgctccagataatcttgtccctgctcctcacttccacaggggacattgtgaggccactggcatggatgctatgcaccccaccctttgctggccatattcctcctgtccccatgctgtggctcatgccaacctagcaataaggagctctggagagcctgcacctgcctcccgctcgcctatatctgctgcccagaggcctgtctcgcacaggggtctcgccactgccaaagactcccaggaagtcaaagactcccagtaatccactagcaaatggaactctgtaacgccatcataacaagagtggccactctccgcgtgcacaggtatgaaatataaatccttacacacacacacacacacaccctcggctcagccacggcactcgccttttatacagcgtcatcgctggacagccaactagaactctgcatcctgtcacaggaagcacctcataagaaggaatggggagggaaggcagtcgccttgttttcagaccttagccgaattc

SEQ ID NO: 20 - protein, 492 aa, Mus musculus
VSRDDDLLLPYPLARRRPSRDCARVRSGSPEQESWPPPPLATHEPRAPSHHAAVRTFVSHFEGRAVAGHLTRVADPLRTFSVLEPGGAGGCGGRSAAATVEDTAVRAGCRIAQNGGFFRMSTGECLGNVVSDGRLVSSSGGLQNAQFGIRRDGTIVTGSCLEEEVLDPVNPFVQLLSGVVWLIRNGNIYINESQAIECDETQETGSFSKFVNVMSARTAVGHDREGQLILFHADGQTEQRGLNLWEMAEFLRQQDVVNAINLDGGGSATFVLNGTLASYPSDHCQDNMWRCPRQVSTVVCVHEPRCQPPDCSGHGTCVDGHCECTSHFWRGEACSELDCGPSNCSQHGLCTAGCHCDAGWTGSNCSEECPLGWYGPGCQRPCQCEHQCFCDPQTGNCSISQVRQCLQPTEATPRAGELASFTRTTWLALTLTLIFLLLISTGVNVSLFLGSRAERNRHLDGDYVYHPLQEVNGEALTAEKEHMEETSNPFKD

SEQ ID NO: 21 - DNA, 9792 bp, Mus musculus
caggctcgggacttactataacacaggacacttgtcacctgaaagcttgagtcagtcagttattatggtctgtgtgtgagatacaagtgggtgcataggcagtggtgcacacatgtagatcagactttctacagccaattctcttcttcctcctctccatgggttcagggtcttcatctcaggttgcacagcgagttcatttatgtgctgtgccatctcgccagtcgttcctatatcctagaggaaaactagtttcttctggtcaagaggaggaaagagtggagacctgtcattctaagatacccaaaacagggccaggttggggacctgtgcctttaatcccatcacttggggattaggtagaagcaagaggctctagaccagtctacacactgaatttcaagccagcctacctataaatcagagaccctgcttcaaaaataaaattaaacaaaaacgaagataaaccaagctacccaaaacacaagagttaatccagtcagacaggtctagcaaatgctaggatgaaaggtgtgcaccaccacgagtgggctgcaagcctctctctctctctctctctctctctctctctcgtttgttttgtttttcgagacaaggtttctctgtgtagccctggctgtcctggaactcactctgtagaccaggctggcctcgagcttcactcttaaaagttcctcttcctcctcctccatcttttcctcctcttaccccctaggctccttttcctcttcttgtctttcagataaagtctcaagtagtccagactggtctcaaactaactaactagccaagaatagccaacctcttaacttccgattctcctgcctctgctgaatgctggggttgtggcgtgggccaccacttctggtttgtgcaacacagaaggaactagggctttaagcacgagaagcaagttctgtacagacttacacaggcccagcatctgttcttgcaattttctgtaagtttgacataatatgagaataaaaagctatctatctcccttccagccttaccctctctgatggaattcgaatgcgtaatcaaagcacccaacagcctggcctgaaatcacgtggggcaagcccacgtgaccggagcaccaatccaatatggcggcgcccagggggcccgggctgttcctcatacccgcgctgctcggcttactcggggtggcgtggtgcagcttaagcttcgggtgagtgcaagccgccggggccagcctggctggggtccacctttcctgagcgctctcaggcacagccctccgacctcacgatcgccccgtccctgcagggtttcccgcgacgatgacctgctgctgccttacccactagcgcgcagacgtccctcgcgagactgcgcccgggtgcgctcaggtagcccagagcaggagagctggcctccgccacctctggccacccacgaaccccgggcgccaagccaccacgcggccgtgcgcaccttcgtgtcgcacttcgaggggcgcgcggtggccggccacctgacgcgggtcgccgatcccctacgcactttctcggtgctggagcccggaggagccgggggctgcggcggcagaagcgccgcggctactgtggaggacacagccgtccgggccggttgccgcatcgctcagaacggtggcttcttccgcatgagcactggcgagtgcttggggaacgtggtgagcgacgggcggctggtgagcagctcagggggactgcagaacgcgcagttcggtatccgacgcgatggaaccatagtcaccgggtgaggaggcagggagccccggggctgtagagggcaaagggtctctgatgttctttcagagccatgcctccgagtccaggtccctaaccaaacttcctgtctttcttcttccgagtaatgacgctgacaccttccttcctttaagtttattcatgtgccactgaataatctgtgatcaggccgtgtgtggggacttggggaggcgaccgtgagcctgaacacagtttgtgccctagtgaactttgtgtagtattagagaaacatttcgtgttcaacgaagccatggaaccaattggaaatagtgtagagtttatggagcagtcccagacagctagctggaggccttttgctgtcctgataaaaatccaggttagacaaggagcttgttgagggcagcctttggaagtttctgtgtttcttgaaatttgacagcagccagagttgacagcaggcaggcaggagtagaaggtagcgccatctggtgttccagttctcttccaaggttccgttttttgccaaggctgggaagtgggctttccccaactcttctcagcccttggttgcaatttctgggcctgcccatgtatctggttcttcatccttcaacatcagccagtgtcaccactgttgatcttaggttttcacagatcctaaaacttctgccagtgaccagcgcctgcagtttctcttccctggctctgtccttcaacctctctacattccagccatctccctagctcctctcttggactccctttcagacttgttgtcatgatcactgtctcagaacccctattgctcctttacaatggtccactgacctgctcacctcctactttttttttttaaatgtgtgtgcatctgtgtgtgcctgaggggagaccagagtttgatttcaaatgtcttctattctcttttcctccatcttattttctaacacaaaatctgaatctagagatcactggttcagttaacctggctggccggtaaaccccagggccctcctgcttccctctgtccaccccaccccagcactaaggctacagtgtgtgctgttccagccagctttctcatgggtgctgaggatctgaacgcaggttcacatgtgtggtgggaaggcttttacccaatgctctgtctttccagcccatcctcccttgttaactgccaaacagctgcctatcctgtccatgtgtagctcactgctacttcttttattatgaggtcagcacatgttactaaagatggcaagagaagaaggttctttcattgtgtcatagctatagctcaggaggaattttatttcctgtgtaggcacacaggagagcatcttccagctcacactccaactgaactaactgaacacctgcctatatatccaaagaaggggtgtcagtgccaatcacagcacacctccagtgcaaatgaaggtttgtgtttgcaccaatcacagccttgcctcttttagcatgcatcacaacaaagtcctcctagactatcaggggatatgctctcttggccaaggtaggaatagttgcagtgtcatctggcacaaaccatttcaaacggcctggctgaggttatgccttcgggaacctgaagtctttgtgtggttgtctccaagtgtctgtggagctccaggcggctggtgctgacagacgctttgtctagttggctgtttgacttttgcttaagcagccagggcagtagagtctaacagatgctaatttcaggatcaggaagactgtagaaaaatgagcatcaagaagcccctggtacccaaagctgctcttgccaatgagtgaacctctgccttcccgcttccaggtcctgtcttgaagaagaggttctggatcccgtgaatccgttcgtgcagctgctgagcggagtcgtgtggctcatccgcaatggaaacatctacatcaacgagagccaagccatcgagtgtgacgagacacaggagacaggtcaggaagcacaggtgttctgttttatttgtattaggttttgatttgtttattttgtgcatgcagcgggtgcatgcatgctcctttcctttcgccatgtgagtcctgagtattgaactcagactgttaagtgtgatgggaggcactttacccactgagccactttcccagccctcagcatcagctttcttcagacccaggaacagtgtgagtgggttattctttagtgttcccaaacatttactgagcagctatttactgtttagcactatggtgagagtcctagggattcagtcttatgtagaatatagaaggagaatccttggcaataagctggaaaattgtgacaagtgccaagaaagaaacaggagaaaggggaccggtggggaccagaagcacaggtatgaggaaagtgcctgcagatttgctgtatggtggcctccacatggcctaggagtttgtcataaatgcagagccatgagtccaccctccctatacctcccatccagaaaccactggttaaatcctaacaacttgggtgtgcaggcactcccttggtgactctgatggacactcaaggtcaagggccacttggggatgggctgatgagttggcttggtcagtaaagtatttgccttgaaagtgtgaggacctgagttggagccccagaaagaaacattaaaagccaagtgctgggatgcacacttgcattcccagggatggagctggaaggcagggataggcagatccacggccacacggtgatattctaagctaacaagagacctgtctcacacagaaagtgggtggcacctgaggaccaacacccagggttatcctctgacgtacctccagagtggaaaatactggggtggtggaaaaggacactttggtcctgggaatctggctattcagggtatagtgtagagggagagggagactcaagaggctgtctttgagtcaaaggaacaagctatcagaagaactcagggcagaggcctgtggttcccaggctcagggcagccttcaaggccctaggcagagagtagctgctgggtgaacaagtacagaagtgaggcctggggcctcaggcaaggcctgtgaaatccttccaccaacatagaagtttctggagactgagatcacatgaagtgcttctggctgtggcatggaagctcactggaggtggagctgggatgtggctcagtgatccagtgcttgccacacgtgcacgagggaaggagccatcaaaagagagaaagtcgggagacctgaggggtcccctggagagctgggtaaccaccccgggcccttctcctttaggttcttttagcaaatttgtgaatgtgatgtcagccaggacagccgtgggtcatgaccgtgaggggcagcttatcctcttccatgctgatggacagacggaacagcgtggtgagtcccaggaaccttggggctgtttgcacttcagccaccctacctttccagtcggttctggggtattggtgggacaagacagctttccggccattttggaagtttcatctggaggcaatagcatttacctactagtgaaagaagccagttaagccagagaccacaggggctcaagctgcataccccctctgcacagccttaacctatgggagatggcagagttcctgcgtcaacaagatgtcgtcaatgccatcaacctggatggaggcggttctgctacttttgtgctcaatgggaccctggccagttacccttcagatcactggtaagaacccttgagccacctttgtggctctctcagactgtctcactcagtcaatactgagaccctgttgtgtgccaggccctgggtatccaaaagtgagcagaagagccgagatctcttccctcagggtgctgcacagcccatccctggaaacctgagacaggtcaggaaaggcctccctgaggacagtgaagtaagacctgaggagatggctggccggggttgagagagcctttaccggaagacaaactgtacgcaatggggaaatccgctaagtggcccagggagaggctggagctatagctcaggaggaaaagtacttgcctcgcaagcgaaggacctgagtttaaactccaaaacccatataaaaagccagatacgagcaagtggcacatgcttgcagtcccagccttgttgaggaagagtcaggtgaatcctgaccctctggccagccagcctagcctactttttggcaaggtccaggccagcgagaaagataaataaaataaagttttaaatgacatgtatctaaggttgtcctgactccatatgcgcacgcacgcatgcacgcacgcacaactggcagaatggaaagggaggcaaactggacagcctttataggctgcggcagggaccagcaccaaggcctagacctcgtctcacagtgaatcccccacagccaggacaacatgtggcgctgtccccgccaagtgtccactgtggtgtgtgtgcatgaaccgcgctgccagccacccgactgcagtggccatgggacctgtgtggatggccactgtgaatgcaccagccacttctggcggggcgaggcctgcagcgagctggactgtggcccctccaactgcagccagcatgggctgtgcacagagagtgagtggggagcccacaggagggtggtgctctggcgggaccccagctcgcccatgctagactcccgcctgtgtccttacccagcctctgtggtcttgctttggtagctggctgccactgtgatgctgggtggacaggatccaactgcagtgaaggtgagagctgcctgcaaacactcctggagagggtggcctggctgcacgcagctggtatgacgccttcgtccctccttctggcttggaacttaccttcagagccttttctcatttcgcatgtggatacccgatgttctacctactgaaagagcccacaagtaggaagccagattttcagtattgtcactcaactctaaggaccaatagcaaaaaaacaaagtggccacgcccctgagggagatccaccaaagtccttaactcctggaaagcagctcctggtgatcctaggcatgggtagggtggtttcagcatcagctcagtggagttcccattcataatttcttcatccttttaaggtcataagttctagagcccaccttaaatctaggcagtattcttggtgtttatctgagacaaagtcttatacagcccacgcagttctctaacttagtatgtaaccgagaatggcctcaagcaacctgcttcctcctttcaagcgctgggattataggcatagcaccaacttatagggtgctagaagtcaaacccagggccctatgtatatgcagcaagcactctagaaactggaacacagccctgtttgcagcccggttaccttggagggttgggtcccagggatctgagggcatctccttcagcatggccatgtgcacacccaggagccaggctgtctgtgacaggagaccatgccacccaaggtgagacctccctgccaccatctcctctccacagagtgtcctctgggctggtatgggccaggttgccagaggccctgccagtgtgagcaccagtgtttctgtgacccgcagactggcaactgcagcatctcccaaggtatgcggccttaaaggttcttgagctgggagcccttggggcaggtctggggtaggtggactctccccagcccttctttctggtgtcttgcagtgaggcagtgtctccagccaactgaggctacgccgagggcaggagagctggcctctttcaccaggtaagtgttttagcaggcactgagcccctatgtctcatccgtgaggcactagccaggccaggaggtcacaggttaccctctactttgcaagctcagggacagtcacaggtaaaactggcatccaggaaagaccctgagctacccagtggaactcaaaggtagcaggctatgggtgtcatgcctctggctgcagagactccacttagatgctggagcagggccatagagacaggaaggactcaccttatttctgaactcttccgtgtgttcaggctttgtgttgttgttgcttcctttctgctgtttcctgggtttccagctccatccccacagggctcatggaaagaattgtgaagcagggggtgtggctcaattggcagattgattgcctggcatgcagaaagccctaggttcaatccccagcatttcatatcataacccaggcatggtggcatcatgtgcctgtaagtccagcacttgggaggtagaagcagaaaagccacgagtttaagaatgttagggagtcttaggccaacctgggatacctaagacaagagatagatgtagggagatagattgacagacagacagacagacagacagacagacagatcttgagctggaccttctggcacaagcctgtcatcctagctattccaggaagctgaagcaggaagatagcaaattcaaggccagcttaagccacagattgagttcaagatcaacctgagcaactttatgaaatcctattataacataaaaagtaggggtgggaggttaggctgtagctcagtggtagagtgattgcctagcacgcacaagacccaggttcaattcccagtactgcaaaaaatatattaggaaccccctaaaagcagtaacattcacattagatgtgtgtgtgtgtgtgtgtgtgtgtgtgtgtgtgtgtgtgtgtgtgtgtgtgttttgttgggtatttatttcatttacatttccaatgctatcccaaaagtcccccacatcctcccccacccaccaccttgtttttttttttttttttttttttttttttgacctgaaactcacaggttaggttagacaagctgactggtgagctccaacttccaacgtaccatcatgcctggcttttgttttggtgtctctgtgtaaccctggatgtcctggagctctctctgtagaccagcctggccttaaactcacagaaacccacctgtttctgcctcccatgtgctgggattaaaggcgtgtgccacctcacccagccctgctggacttaaattgggtcttcattttataagacaagcatgagctaattccccagttcctaaaatgtttttaacatccttaaacatcagagactgtctgtggtattccctccatgtgtcttcagtatacctactcccctccctgcctactgggttcaacatgcccagtttgggttctggctgcctgcccccactcaagactctcttttccatctcaggaccacctggctagccctcaccctgacactaattttcctgctgctgatcagcactggggtcaacgtgtccttgttcctgggctccagggccgagaggaaccggcacctcgacggggactatgtgtatcacccactgcaggaggtgaacggggaagcgctgactgcagagaaggagcacatggaggaaactagcaaccccttcaaggactgaagagctgccccaacggcatgctccagataatcttgtccctgctcctcacttccacaggggacattgtgaggccactggcatggatgctatgcaccccaccctttgctggccatattcctcctgtccccatgctgtggctcatgccaacctagcaataaggagctctggagagcctgcacctgcctcccgctcgcctatatctgctgcccagaggcctgtctcgcacaggggtctcgccactgccaaagactcccaggaagtcaaagactcccagtaatccactagcaaatggaactctgtaacgccatcataacaagagtggccactctccgcgtgcacaggtatgaaatataaatccttacacacacacacacacacaccctcggctcagccacggcactcgccttttatacagcgtcatcgctggacagccaactagaactctgcatcctgtcacaggaagcacctcataagaaggaatggggagggaaggcagtcgccttgttttcagaccttagccgaattc

(19) United States
(12) Patent Application Publication
(10) Pub. No.: US 2003/0124652 A1
(43) Pub. Date: Jul. 3, 2003

(54) Methods of producing high mannose glycoproteins in complex carbohydrate deficient cells

(75) Inventor: William M. Canfield, Oklahoma City, OK (US)

Correspondence Address: OBLON, SPIVAK, MCCLELLAND, MAIER & NEUSTADT, P.C., 1940 DUKE STREET, ALEXANDRIA, VA, 22314, US

(73) Assignee: NOVAZYME PHARMACEUTICALS, INC., Oklahoma City, OK

(21) Appl. No.: 10/023,889

(22) Filed: Dec. 21, 2001

(51) Int. Cl. C12P 21/02; C12N 5/06; C07H 21/04; C12N 9/36; A61K 35/78

(52) U.S. Cl. 435/69.1; 435/320.1; 435/206; 435/325; 536/23.2; 424/731

(57) ABSTRACT

The present invention provides a method for producing high mannose glycoproteins in complex carbohydrate deficient cells and the glycoproteins obtained therein.

### BACKGROUND OF THE INVENTION

1. Field of the Invention

The present invention provides a method for producing high mannose glycoproteins in complex carbohydrate deficient cells and the glycoproteins obtained therein.

2. Discussion of the Background

In the area of enzyme replacement therapy many proteins are produced in recombinant mammalian cells to facilitate proper processing to better provide for specificity and activity. Following or concurrently with translation of the messenger RNA into proteins, the protein is guided through the endoplasmic reticulum and Golgi apparatus where they undergo various modifications, including the attachment of complex oligosaccharides (e.g., those containing galactose). The specific post translational modifications may vary depending on the species of the host cell and accordingly non-native protein expression typically suffers from non-native glycosylation patterns.

The enzymes that are modified with such complex oligosaccharides are cleared rapidly by the liver due to the presence of the carbohydrate and particularly high affinity Gal-GalNac specific lectin, i.e., asialoglycoproteins receptor (Breitfield et al (1985) Int. Rev. Cytol. 97:47-95). The net result of the liver clearance is a significant reduction in the bio-availability of the administered protein. Terminal galactose residues are responsible for the clearance by the liver, which bind to asialoglycoprotein receptors on the surface of liver cells. Additionally, Chinese Hamster Ovary cells, which are commonly used to produce recombinant glycoproteins, utilize N-glycolylneuraminic acid. Preformed antibodies to N-glycolylneuraminic acid are believed to be responsible for serum sickness in humans following administration of heterologous serum. Thus, elimination of the complex type oligosaccharides from the glycoprotein can provide a safer and more effective starting material for the manufacture of highly phosphorylated GAA for use in a replacement therapy.

Accordingly, there is a great clinical need, particularly in enzyme replacement therapies to produce proteins without, or at minimum little, complex carbohydrates on the surface of recombinant enzymes utilized in enzyme replacement therapies.

Lectin resistant cell lines, in general, are known (Stanley (1983) Meth. Enzymology 96:157-189; Gottlieb et al (1974) Proc. Nat. Acad. Sci., U.S.A., 71(4):1078 1082; Stanley et al (1990) Somat Cell Mol Genet (3):211-223). Characteristics of lectin resistant cell lines include the production of proteins in the absence of sialic acid residues, galactosamine and other carbohydrate moieties on the terminal oligosaccharide structure of the modified protein yielding only high mannose structures. Generally, lectin resistant cells have altered surface carbohydrates resulting in complex N-glycan blockage (Stanley (1983) Meth. Enzymology vol. 96:pp157-184).

One example of such a lectin is Ricin from Ricinus communis or Castor Bean is a galactose-binding lectin with potent cytotoxic effects. Growing CHO cells in the presence of Ricin has been shown to select for cells that are typically resistant to this lectin. One class of the CHO cells that survive this selection process are characterized by their inability to synthesize complex type oligosaccharides on their glycoproteins and by the presence of only high mannose type oligosaccharde side chains. (Stanley (1983) Meth. Enzymology vol. 96:pp157-184).

To practically effectuate blockage of complex carbohydrate formation on proteins for enzyme replacement therapy—theoretically it should be possible to transform a lectin resistant cell line with an expression construct carrying the gene encoding the enzyme. For example, α-glucosidase, which is a lysosomal hydrolase whose absence in human patients results in the lysosomal storage disorder Pompe's disease, in order to achieve highly effective enzyme replacement of lysosomal hydrolases proper phosphorylation by N-Acetylglucosamine-1-phosphotransferase (“GlcNAc-phosphotransferase”) and N-acetylglucosamine-1-phosphodiester α-N-Acetylglucosaminidase (“phosphodiester α-GlcNAcase”).

GlcNAc-phosphotransferase catalyzes the first step in the synthesis of the mannose 6-phosphate determinant, which is required for the intracellular targeting of newly synthesized acid hydrolases to the lysosome. A proper carbohydrate structure greatly facilitates the efficient phosphorylation by GlcNAc-phosphotransferase. In the case of lysosomal enzymes the carbohydrate structure coupled to phosphorylation is necessary for the synthesis of a mannose-6-phosphate signal on the GAA molecule is a high mannose N-glycan.

Prior to the present invention, lectin resistant cell lines were reported and such cell lines were reported to have defect(s) in the glycosylation pathways (Stanley (1983) Meth. Enzymology vol. 96:pp157-184). Thus, one approach to producing glycoproteins with a reduction or loss of complex carbohydrates would be to introduce a gene expressing the glycoprotein into one of the lectin resistant cell lines known previously. Following introduction and expression, the user could recover the glycoprotein, presumably with reduced complex carbohydrates on its' surface. However, in attempts to transform a lectin resistant cell line in order to express a non-native glycoprotein, e.g., acid α-glucosidase, the amount of protein expressed and thus recovered was very poor thereby having little practical utility.

The present inventors have discovered quite unexpectedly that when a mammalian cell is transfected to express a glycoprotein of interest is subjected to lectin selection, one is able to obtain both high levels of glycoprotein expression coupled with a reduction in complex carbohydrates on the glycoproteins' surface are observed. Accordingly, one aspect of the present invention is a method of producing non-native glycoproteins having reduced complex carbohydrates structures.

As discussed above, a certain class of glycoproteins, lysosomal hydrolases effect lysosomal function and when deficient or malfunctioning can result in a variety of lysosomal storage disorders. These lysosomal hydrolases require efficient phosphorylation and removal of the N-acetylglucosamine group on the surface of lysosomal hydrolase for most efficient targeting to the lysosome organelle. Those hydrolases containing certain oligosaccharide structures such as GlcNAc-2 Man-7 isomer D2 are found to be better substrates for phosphorylation mediated by GlcNAc phosphotransferase and phosphodiester α-GlcNAcase.

It was believed prior to the present invention that treating cells with either deoxymannojirimycin (DMJ) or kifunensine (Kif) results in the inhibition of glycoprotein processing in those cells (Elbein et al (1991) FASEB J (5):3055-3063; and Bischoff et al (1990) J. Biol. Chem. 265(26):15599-15605). These inhibitors block complex sugar attachment to modified proteins. However, if sufficient DMJ and Kif are utilized to completely inhibit glycoprotein processing on lysosomal hydrolases, the resultant hydrolases have mannose-9 structures, which are not the most efficient substrates for the GlcNAc phosphotransferase enzyme. Man-9 glycan structures cannot be bis-phoshphorylated and therefore do not provide the highest affinity ligand.

The present inventors have taken the method of glycoproteins with reduced complex carbohydrates in lectin resistant mammalian cells and treated the lectin resistant cells with DMJ and Kif on the basis of further inhibiting the glycosylation pathway in those cells. The inventors have surprisingly discovered that not only is the glycosylation pathway further inhibited but the coupling of the lectin resitant cells with DMJ/Kif treatment yields lysosomal hydrolase glycoproteins having a mannose structure that is the preferred substrate for the aforementioned lysosomal phosphorylation enzymes. Accordingly, another aspect of the present invention is a method of producing non-native glycoproteins, in particular lysosomal hydrolases, with high mannose structures.

### SUMMARY OF THE INVENTION

Accordingly, an object of the present invention is to provide methods of preparing glycoproteins with reduced complex carbohydrates by expressing the glycoprotein in cells, culturing the cells in a lectin in an amount sufficient to obtain a lectin resistant cell and collecting the glycoprotein produced from the cell.

In a preferred embodiment the glycoprotein is a lysosomal hydrolase.

Another object of the invention is to treat the glycoprotein with GlcNAc-phosphotransferase to transfer an N-acetylglucosamine-1-phosphate.

Another object of the invention is to further treat the glycoprotein to remove the N-acetylglucosamine moiety with phosphodiester α-GlcNACase.

Another object of the invention is to provide treatment methods for patients suffering from a lysosomal storage disease with lysosomal glycoproteins produced by the methods disclosed herein.

### BRIEF DESCRIPTION OF THE DRAWINGS

FIG. 1: Phosphorylation of rh-GAA produced from cells cultured in the presence of DMJ or Kif alone. The y-axis depicts the per amount of [³²P] incorporation, the X axis represents the amount of inhibitor added to the cultured cells, referring to Table 1 for the amounts used. is the DMJ curve, is the Kif curve.

FIG. 2. Phosphorylation of rh-GAA produced from cells cultured in the presence of rh-GAA with the combination of DMJ and Kif. The y-axis depicts the per amount of [³²P] incorporation, the X axis represents the amount of inhibitor added to the cultured cells, referring to Table 1 for the amounts used. is DMJ, is Kif.

### DETAILED DESCRIPTION OF THE INVENTION

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausebel et al (eds.), John Wiley & Sons, New York (2001) and the various references cited therein.

“Isolated” means separated out of its natural environment.

“Polynucleotide” in general relates to polyribonucleotides and polydeoxyribonucleotides, it being possible for these to be non-modified RNA or DNA or modified RNA or DNA.

The term “nucleotide sequence” as used herein means a polynucleotide molecule in the form of a separate fragment or as a component of a larger nucleic acid construct that has been derived from DNA or RNA isolated at least once in substantially pure form (i.e., free of contaminating endogenous materials) and in a quantity or concentration enabling identification, manipulation, and recovery of its component nucleotide sequences by standard biochemical methods. Such sequences are preferably provided in the form of an open reading frame uninterrupted by internal non-translated sequences, or introns that are typically present in eukaryotic genes. Sequences of non-translated DNA may be present 5′ or 3′ from an open reading frame where the same do not interfere with manipulation or expression of the coding region.

The term “nucleic acid molecule” as used herein means RNA or DNA, including cDNA, single or double stranded, and linear or covalently closed molecules. A nucleic acid molecule may also be genomic DNA corresponding to the entire gene or a substantial portion therefor to fragments and derivatives thereof. The nucleotide sequence may correspond to the naturally occurring nucleotide sequence or may contain single or multiple nucleotide substitutions, deletions and/or additions including fragments thereof. All such variations in the nucleic acid molecule retain the ability to encode a biologically active enzyme when expressed in the appropriate host or an enzymatically active fragment thereof. The nucleic acid molecule of the present invention may comprise solely the nucleotide sequence encoding an enzyme or may be part of a larger nucleic acid molecule that extends to the gene for the enzyme. The non-enzyme encoding sequences in a larger nucleic acid molecule may include vector, promoter, terminator, enhancer, replication, signal sequences, or non-coding regions of the gene.

Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Other control or regulatory sequences can be employed as is known in the art. Exemplary expression vectors for use in mammalian host cells are well known in the art.

Methods of introducing, transducting or transfecting mammalian cells are well within the knowledge of the skilled artisan. Examples of such methods include calcium phosphate-mediated, liposome-mediated, Dextran-mediated, and electroporation. These and other methods are described in, for example, Sambrook et al (2001) Molecular Cloning: A Laboratory Manual, Cold Spring Harbor Laboratory Press, NY and Current Protocols in Molecular Biology (2001) and Ausebel et al (eds.), John Wiley and Sons, Inc, New York.

According to the present invention, the glycoproteins may be produced by the recombinant expression systems described above. The method comprises culturing a host cell transformed with an expression vector comprising a DNA sequence that encodes the glycoprotein under conditions sufficient to promote expression of the glycoprotein.

“Polypeptides” are understood as meaning peptides or proteins which comprise two or more amino acids bonded via peptide bonds.

“Glycoprotein” as used herein means proteins that are endogenously modified to carry one or more carbohydrate moieties on the protein. Within the context of the present invention, lysosomal hydrolase glycoproteins are preferred. Examples of lysosomal hydrolases include α-glucosidase, α-L-iduronidase, α-galactosidase A, arylsulfatase, N-acetylgalactosamine-6-sulfatase or β-galactosidase, iduronate 2-sulfatase, ceramidase, galactocerebrosidase, β-glucuronidase, Heparan N-sulfatase, N-Acetyl-α-glucosaminidase, Acetyl CoA-α-glucosaminide N-acetyl transferase, N-acetyl-glucosamine-6 sulfatase, Galactose 6-sulfatase, Arylsulfatase A, B, and C, Arylsulfatase A Cerebroside, Ganglioside, Acid β-galactosidase G_{M1} Galglioside, Acid β-galactosidase, Hexosaminidase A, Hexosaminidase B, α-fucosidase, α-N-Acetyl galactosaminidase, Glycoprotein Neuraminidase, Aspartylglucosamine amidase, Acid Lipase, Acid Ceramidase, Lysosomal Sphingomyelinase and other Sphingomyelinases.

The term “biologically active” as used herein means an enzyme or protein having structural, regulatory, or biochemical functions of a naturally occurring molecule.

“Complex carbohydrates” as used herein means contains monosaccharide other than GlnAc and mannose (Kornfeld, R and Kornfeld, S. (1985) Ann Rev Biochem 54:631-664).

The phrase “reduced complex carbohydrates” as used herein means a glycoprotein with reduced complex carbohydrate structures on its' surface, where the term reduced means less than the amount relative to the amount of complex carbohydrates found on the same protein in a cell not modified or treated as described herein for the present invention. Likewise, “complex carbohydrate deficient” means that glycoproteins, and cells that produce the glycoproteins, which do not have complex carbohydrates detectable by methods known to the skilled artisan.

The phrase “high mannose oligosaccharides” as used herein means containing only core GlcNAc and mannose (Kornfeld, R and Kornfeld, S. (1985) Ann Rev Biochem 54:631-664)

Levels and/or types of complex carbohydrate structures can measured using known methods. For example, glycoproteins and their associated oligosaccharides can be characterized using endoglycosidases to differentiate between high mannose and complex type oligosaccharides (Maley et al (1989) Anal. Biochem. 180:195-204). Peptide-N⁴-(N-acetyl-β-glucosaminyl)asparagines amidase (PNGaseF) is able to hydrolyze asparagines-linked (N-linked) oligosaccharides at the β-aspartylglycosylamine bond to yield ammonia, aspartic acid and an oligosaccharide with an intact di-N-acetlychitobiose on the reducing end. The specificity of PNGase is broad because high mannose, hybrid, di-, tri- and tetraantennary complex, sulfated and polysialyl oligosaccharides are substrates. Additionally, endo-β-N-acetylglucosaminidase H (EndoH) effectively hyrdolyzes the chitobiose unit in hybrid- and mannose-containing N-linked oligosaccharides possessing at three mannose residues, providing that the α1,6-mannose arm has another mannose attached. Complex oligosaccharides are resistant to EndoH digestion.

To characterize the type of N-linked oligosaccharides present in glycoproteins, an aliquot of protein can be digested with PNGaseF (0.5% SDS, 1% β-mercaptoethanol, 50 mM NP-40, 50 mM Sodium Phosphate, pH 7.5) or EndoH (0.5% SDS, 1% β-mercaptoethanol, 50 mM Sodium Citrate, pH 5.5) under reducing conditions. The native and digested proteins are then analyzed by SDS-polyacrylamide electrophoresis under reducing conditions and the relative mobilities compared. If the glycoprotein contains only high mannose oligosaccharides the PNGaseF and EndoH treated samples will have a greater mobility than the untreated protein. The EndoH treated protein will have a slightly higher molecular weight due to the single remaining N-acetylglucosamine at each N-linked glycosylaton site. IF a glycoprotein contains only complex oligosaccharides, the EndoH treated protein will not have a shift in migration compared to the untreated protein. If there are both complex and high mannose oligosaccharides, then EndoH treated protein will be smaller than the non-treated glycoprotein but larger than than the PNGaseF treated protein. The difference will be greater than that which can be accounted for by the remaining N-acetylglucosamine.

Likewise, Neutral and amino sugars of glycoproteins can be analyzed by high-performance anion-exchange chromatography. Composition analysis is used to determine the type and amount of monosaccharides in glycoproteins and to quantify amounts in structural studies. Monosaccharides are released by acid hydrolysis with 4 NTFA at 100° C. for 4 hours in polypropylene tubes washed with 6N HCl. This hydrolysis method results in a significant recovery of monosaccharides (Bousfield et al (2000) Methods 21:15-39). Following hydrolysis, samples are dried under vacuum and the resulting monosaccharide mixture is separated and quantified using high-performance anion-exchange chromatography (HPAEC) with electrochemical detection. Separating carbohydrates is achieved by converting the normally neutral monosaccharides to anions at a pH greater than their hydroxyl group pKa range of 12-13, using sodium hydroxide as an eluent (Olechno et al (1988) Am. Biotech. Lab. 5:38-50). Both neutral and amino sugars can be analyzed in a single analysis (Lee (1990) Anal. Biochem. 189:151-162). As the negatively charged sialic acids and phosphorylated mannoses are more strongly retained than the neutral and amino sugars, a second method is used that elutes the analytes by increasing the sodium hydroxide to 150 mM and adding 150 mM sodium acetate to the eluent. The harsg conditions imposed by these methods require nonmetallic flow path and this was accomplished by the use of polyether ketone (PEEK) extensively through the instrument flow path. Detection of the monosaccharides employs triple pulse amperometry (Lee (1990) Anal. Biochem. 189:151-162). The pulsed amperometric detector gold electrode is held at an analytical potential for a brief period, 100 to 200 ms. At this potential, 1% of the monosaccharide sample in the flow path is oxidized and the current carried by the resulting anions is measured at a reference electrode. Fouling of the gold electrode is eliminated by the cleaning cycle that follows immediately after analyte sampling. A strong oxidizing potential is applied to completely oxidize any adsorbed materials on the gold electrode surface followed by a reversal of potential to renew the gold surface. The maximum sensitivity of the standard instrument is about 10 pmol. Routine measurements are accomplished with 30 μg samples. Molar amounts are determined by comparing peaks area against standard 5 point curve of know molar amounts of each monosaccharide.

In the present invention any mammalian cell can be utilized, primary or established. Preferably, the mammalian cell is an established cell line that proliferates in culture and is amenable to selection as described herein. Examples of such cells include HeLa, 293T, Vero, NIH 3T3, Chinese Hamster Ovary, and NS0.

Mammalian cells can be cultured in dishes, plates, and flasks in the appropriate medium in accordance with standard cell culture protocols (Sambrook et al (2001) Molecular Cloning: A Laboratory Manual, Cold Spring Harbor Laboratory Press, NY and Current Protocols in Molecular Biology (2001) and Ausebel et al (eds.), John Wiley and Sons, Inc, New York). As recognized by the skilled artisan the type of vessel and specific culture conditions will vary depending on the specific cell type, whether the cell is typically cultured in suspension, adherent or in a co-culture with one or more cells.

The term “lectin” as used herein includes those compounds that are known to be hemagglutinating proteins. Typically, the proteins are isolated from plant seeds and bind to cells via cell surface carbohydrate receptors. Lectins are often toxic to cells in certain doses, which varies depending on the cell type and lectin studied. Examples of lectins include ricin, concanavalin A, erthroglutinin, lymphoagglutanin, and wheat germ agglutinin. Preferably, the lectin is ricin. Ricin binds to complex oligosaccharides and is lethal to cells. In cells found to be lectin resistant mutants, the carbohydrate profile of glycoproteins is altered.

The lectin may be administered to cells by mixing with cell culture media prior to addition to the cells, added to the medium in which the cells are already being cultured, coated onto the culture vessel and/or combinations of these. Additionally, the lectin may be added several times during the culturing process and/or concurrently with or independently of changing the cell culture media.

The amount of lectin to be employed should be at least an amount which when applied to the cells in culture will have a toxic effect on some of the cells while not killing all of the cells. Accordingly, “lectin resistant cells or lectin resistant mammalian cells” means those cells that are not susceptible to lectin toxicity at concentrations of lectin applied to the cells in culture. The skilled artisan will recognize that the amount of lectin employed in the present invention will vary depending on the specific cell type chosen and lectin employed for the selection. Following the addition of lectins to the cell culture, the cells are observed for a period of time to identify those cells which exhibit resistance to lectin toxicity. Identification of viable cultured cells is within the knowledge of the skilled artisan, for example, substrate attachment, visual inspection by microscopy or other common methods of determining cell viability can be used.

Those cells that are found to be resistant to the lectin can be individually cloned and expanded. Alternatively, the resistant cells may be pooled and expanded. The amount of lectin to be employed can be determined using a lectin cell kill curve. The lectin kill protocol may be performed as follows. Obtain 3 confluent T150 flasks of cells, remove the media and was the cells twice with PBS. Trypsinize the cells in 3 ml Trypsin EDTA and remove immediately with a pipette. Incubate for 5 minutes at 37° C. and resuspend the cells in 10 ml complete DMEM. Count the cells with a hemacytometer. Centrifuge the cells at 1000 RPM and aspirate off the media. Wash the cells in 10 ml DPBS twice and resuspend 25 million cells in 25 ml of serum-free DMEM. Add a range of lectin to be tested, for example at least about 0.1 μg/ml to at least about 20 μg/ml, including 0.2, 0.3, 0.4, 0.5, 0.95, 1.0, 1.10, 1.25, 1.35, 1.50, 1.65, 1.70, 1.75, 2.0, 2.5, 5.0, 6, 7, 8, 9, 10, 11, 13, 15, 17, 19 μg/ml and all values there between. Invert to mix, incubate at 37° C. for 1 hour and resuspend the cells in 1 liter of Selection DMEM. Identify at what concentration either all the cells die or where cell clones can be identified exhibiting lectin resistance (i.e., are viable in the selection medium). When the cells are transfected with a glycoprotein having complex oligosaccharides at a high level, the amount of lectin is preferably chosen to be sufficient to bind all of the complex oligosaccharides.

To produce proteins with high mannose glycoproteins, the lectin resistant cells expressing a recombinant glycoprotein or lysosomal hydrolase are exposed to both DMJ and Kif to further inhibit glycoprotein processing. DMJ and Kif may be first mixed together prior to adding to the culture media, added separately at the same time, and/or added separately at different times. Preferably, DMJ and Kif are mixed together prior to addition to the culture medium. DMJ and Kif may be administered to cells by mixing with cell culture media prior to addition to the cells, added to the medium in which the cells are already being cultured, coated onto the culture vessel and/or combinations of these. Additionally, DMJ and Kif may be added several times during the culturing process and/or concurrently with or independently of changing the cell culture media.

To determine the concentration of DMJ and Kif to be added, two tests may be performed: (1) the concentration of the inhibitor can be varied in the culture media of the cells overexpressing the glycoprotein, isolate the glycoprotein, and analyze the predominate types of oligosaccharides presents as described herein, e.g, using EndoH digestion, glyconase; and/or (2) the susceptibility of various oligosacchardies on lysosomal enzymes to be phosphorylated by GlcNAc-phosphotransferase and phosphodiester α-GlcNAcase can measured as described herein.

DMJ is preferably added to the culture in the amount of at least about 0.1 mM to about 10.0 mM, including, 0.5, 0.75, 1.0, 1.25, 1.5, 1.75, 2.0, 2.5, 3.0, 3.5, 4.0, 4.5, 4.75 mM and all values there between.

Kif is preferably added to the culture in the amount of at least 0.01 μg/ml to about at least 10 μg/ml, including 0.25, 0.5, 0.75, 1.0, 1.25, 1.5, 1.75, 2.0, 2.5, 3.0, 3.5, 4.0, 4.5, 5.0, 5.5, 6.0, 6.5, 7.0, 7.5, 8.0, 8.5, 9.0, 9.25, 9.5, 9.75 and all values there between.

DMJ and Kif are added to the cells for a period of time to effectuate glycoprotein processing and the ability to obtain glycoproteins with high mannose structure. In most instances, the DMJ and Kif inhibitors must be substantially present during the culturing, preferably the inhibitors are present at all times during the culturing procedure.

At the appropriate time, the recovery of the glycoprotein can be in either the culture medium, cell extracts, or both depending upon the expression system employed. As is known to the skilled artisan, procedures for purifying a recombinant protein will vary according to such factors as the type of host cells employed and whether or not the recombinant protein is secreted into the culture medium. When expression systems that secrete the recombinant protein are employed, the culture medium first may be concentrated. Following the concentration step, the concentrate can be applied to a purification matrix such as a gel filtration medium. Alternatively, an anion exchange resin can be employed, e.g., a matrix or substrate having pendant diethylaminoethyl (DEAE) groups. The matrices can be acrylamide, agarose, dextran, cellulose, or other types commonly employed in protein purification. Also, a cation exchange step can be employed. Suitable cation exchangers include various insoluble matrices comprising sulfopropyl or carboxymethyl groups. Further, one or more reversed-phase high performance liquid chromatography (RP-HPLC) steps employing hydrophobic RP-HPLC media (e.g., silica gel having pendant methyl or other aliphatic groups) can be employed to further purify the enzyme. Some or all of the foregoing purification steps, in various combinations, are well known in the art and can be employed to provide an isolated and purified recombinant protein.

In another aspect of the present invention, the lysosomal proteins produced in either the lectin resistant cells or from the lectin resistant cells treated with DMJ and Kif, the lysosomal proteins are phosphorylated with the lysosomal enzyme GlcNAc-phosphotransferase and phosphodiester α-GlcNAcase. The lysosomal enzyme can be treated in vivo or in vitro, before, during or after various purification or isolation steps.

The lysosomal hydrolases are treated with GlcNAc-phosphotransferase which catalyzes the transfer of N-acetylglucosamine-1-phosphate from UDP-GlcNAc to the 6′ position of 1,2-linked or other outer mannoses on the hydrolase. Methods for treating any particular lysosomal hydrolase with the enzymes of the present invention are within the skill of the artisan. Generally, the lysosomal hydrolase is present in a concentration of about 10 mg/ml and GlcNAc-phosphotransferase at a concentration of about 100,000 units/mL are incubated at about 37° C. for 2 hours in the presence of a buffer that maintains the pH at about 6-7 and any stabilizers or coenzymes required to facilitate the reaction. Then, phosphodiester α-GlcNAcase can be added to the system to a concentration of about 1000 units/mL and the system is allowed to incubate for about 2 more hours. The modified lysosomal enzyme having highly phosphorylated oligosaccharides is then recovered as described herein or methods commonly employed in the art.

In a preferred embodiment, the lysosomal hydrolase at 10 mg/ml is incubated in 50 mm Tris-HCI, pH 6.7, 5 mM MgCl₂, 5 mM MnCl₂, 2 mM UDP-GlcNAc with GlcNAc phosphotransferase at 100,000 units/mL at 37° C. for 2 hours. The modified enzyme is then repurified by chromatography on Q-Sepharose and step elution with NaCl.

The GlcNAc-phosphotransferase and phosphodiester α-GlcNAcase employed in the present invention can be isolated from natural sources such as mammalian, preferably human tissues, isolated from recombinant expression systems, such as cell-free translation or eukaryotic expression systems commonly employed in the art. The GlcNAc-phosphotransferase and phosphodiester α-GlcNAcase enzymes can be prepared simultaneously in the same system, separately using the same systems or can be obtained from different systems.

The GlcNAc-phosphotransferase and phosphodiester α-GlcNAcase enzymes and genes encoding the enzymes may be derived from any mammalian source, preferably human, bovine and porcine, and more preferably human.

The GlcNAc-phosphotrasferase is composed of six subunits: 2 α subunits, 2 β-subunits and 2 γ subunits. The amino acid sequence of the α subunit is shown in SEQ ID NO:4 (amino acids 1-928), the human β subunit is shown in SEQ ID NO:5 (amino acids 1-328), and the human γ subunit is shown in SEQ ID NO:7 (amino acids 25-305, signal sequence is in amino acids 1-24).

In another embodiment, the GlcNAc-phosphotransferase is recombinant GlcNAc-phosphotransferase, which has been engineered to remove the membrane binding domain from the polyprotein containing the α/β subunits and the endogenous proteolytic cleavage site is replaced with a non-endogenous site-specific proteolytic cleavage site such as Furin, Factor Xa, Enterokinase, and Genease. Typically the GlcNac-phosphotransferase is transfected in a cell also expressing the γ subunit. However, in some instances it may be preferable to treat the lysosomal hydrolase with the α/β subunits without prior addition of the γ-subunit. A GlcNAc phosphotransferase that comprises only the α and β subunits reduces substrate specificity which allows the GlcNAc phosphotransferase to catalyze the transfer of N-acetylglucosamine-1-phosphate from UDP-GlcNAc to enzymes which is not a natural substrate for the enzyme, e.g, acid β galactocerebrosidase. This modified hydrolase may then be treated with phosphodiester α-GlcNAcase to complete the modification of yielding an enzyme available for targeting tissues via the mannose-6-phosphate receptor. In another embodiment, it may be desirable to treat other glycoproteins with the α/β subunits GlcNAc-phosphotransferase enzyme followed by treatment with phosphodiester α-GlcNAcase to obtain glycoproteins that can be similarly targeted to cells via the mannose-6-phosphate receptor.

The soluble GlcNAc-phosphotransferase protein or polypeptide include the sequences exemplified in this application as well as those which have substantial identity to SEQ ID NO:2.

The partial rat and Drosphila melanogaster α/β GlcNAc-phosphotransferase amino acid sequences are shown in SEQ ID NO: 14 and 16, respectively.

Preferably, the GlcNAc-phosphotransferase polypeptides are those which are at least 70%, preferably at least 80% and more preferably at least 90% to 95% identical to the GlcNAc-phosphotransferase amino acid sequences described herein.

Polynucleotides which encode the α and β subunits of GlcNAc-phosphotransferase or soluble GlcNAc-phosphotransferase mean the sequences exemplified in this application as well as those which have substantial identity to those sequences and which encode an enzyme having the activity of the α and β subunits of GlcNAc-phosphotransferase. Preferably, such polynucleotides are those which hybridize under stringent conditions and are at least 70%, preferably at least 80% and more preferably at least 90% to 95% identical to those sequences

The nucleotide sequence for the human α/β subunit precursor cDNA is shown in SEQ ID NO:3 (nucleotides 165-3932), the nucleotide sequence of the α subunit is in nucleotides 165-2948 of SEQ ID NO:3, the nucleotide sequence of the β subunit is shown in nucleotides 2949-3932 of SEQ ID NO:3, and the nucleotide sequence of the γ subunit is shown in SEQ ID NO:6 (nucleotides 24-95). The soluble GlcNAc-phosphotransferase nucleotide sequence is shown in SEQ ID NO:1. The partial rat and Drosphila melanogaster α/β GlcNAc-phosphotransferase nucleotide sequences are shown in SEQ ID NO: 13 and 15, respectively.

Polynucleotides which encode phosphodiester α-GlcNAcase as used herein is understood to mean the sequences exemplified in this application as well as those which have substantial identity to SEQ ID NO:19 (murine) or SEQ ID NO:17 (human) and which encode an enzyme having the activity of phosphodiester α-GlcNAcase. Preferably, such polynucleotides are those which hybridize under stringent conditions and are at least 70%, preferably at least 80% and more preferably at least 90% to 95% identical to SEQ ID NOS:17 and/or 19.

The phosphodiester α-GlcNAcase protein or polypeptide as used herein is understood to mean the sequences exemplified in this application as well as those which have substantial identity to SEQ ID NO:20 (murine) or SEQ ID NO:18 (human). Preferably, such polypeptides are those which are at least 70%, preferably at least 80% and more preferably at least 90% to 95% identical to SEQ ID NOS:18 and/or 20.

When the glycoproteins are lysosomal hydrolases, following the phosphorylation with GlcNAc phosphotransferase and phosphodiester α-GlcNAcase, the phosphorylated lysosomal hydrolase can be administered to a patient suffering from the lysosomal storage disorder to replace the deficient hydrolase as appropriate. Thus, the present invention also provides methods for the treatment of lysosomal storage diseases by administering an effective amount of the phosphorylated lysosomal hydrolase of the present invention to a patient diagnosed with the respective disease. As used herein, being diagnosed with a lysosomal storage disorder includes pre-symptomatic phases of the disease and the various symptomatic identifiers associated with the disease. Typically, the pre-symptomatic patient will be diagnosed with the disease by means of a genetic analysis known to the skilled artisan.

While dosages may vary depending on the disease and the patient, phosphorylated hydrolase are generally administered to the patient in amounts of from about 0.1 to about 1000 milligrams per 50 kg of patient per month, preferably from about 1 to about 500 milligrams per 50 kg of patient per month. Amongst various patients the severity and the age at which the disease presents itself may be a function of the amount of residual hydrolase that exists in the patient. As such, the present method of treating lysosomal storage diseases includes providing the phosphorylated lysosomal hydrolase at any or all stages of disease progression.

The hydrolase may be administered by any convenient means, conventionally known to those of ordinary skill in the art. For example, the enzyme may be administered in the form of a pharmaceutical composition containing the enzyme and a pharmaceutically acceptable carrier or by means of a delivery system such as a liposome or a controlled release pharmaceutical composition. The term “pharmaceutically acceptable” refers to molecules and compositions that are physiologically tolerable and do not typically produce an allergic or similar unwanted reaction such as gastric upset or dizziness when administered. Preferably, “pharmaceutically acceptable” means approved by a regulatory agency of the Federal or a state government or listed in the U.S. Pharmacopoeia or other generally recognized pharmacopoeia for use in animals, preferably humans. The term “carrier” refers to a diluent, adjuvant, excipient, or vehicle with which the compound is administered. Such pharmaceutical carriers can be sterile liquids, such as saline solutions, dextrose solutions, glycerol solutions, water and oils emulsions such as those made with oils of petroleum, animal, vegetable, or synthetic origin (peanut oil, soybean oil, mineral oil, or sesame oil). Water, saline solutions, dextrose solutions, and glycerol solutions are preferably employed as carriers, particularly for injectable solutions.

The hydrolase or the composition may be administered by any standard technique compatible with enzymes or their compositions. For example, the enzyme or composition can be administered parenterally, transdermally, or transmucosally, e.g., orally or nasally. Preferably, the hydrolase or composition is administered by intravenous injection.

The following Examples provide an illustration of embodiments of the invention and should not be construed to limit the scope of the invention, which is set forth in the appended claims. In the following Examples, all methods described are conventional unless otherwise specified.

### EXAMPLES

Construction of a GS Plasmid for High Level Expression of Human Acid-α Glucosidase—A cDNA encoding human Acid-α-Glucosidase cloned into pcDNA3 (Invitrogen).

The cDNA clone was subcloned into the EcoRI site of the pcDNA3 plasmid following the addition of EcoRI linkers to each end of the cDNA. The plasmid was cleaved with HindIII and EcoRV to generate a fragment encoding GAA containing a HindIII site at the 5′ end and a blunt end at the 3′ end of the cDNA. This fragment was then subcloned into the HindIII-SmaI sites of the pEE14 plasmid (Lonza Pharmaceuticals) to construct the GS expression plasmid. The resulting plasmid, named pBC40.

Generation of a GAA Expressing CHO-K1 Cell Line—CHO-K1 were transfected in 10 cm² culture dishes with the cells at 40% confluency. The cells were grown in Glasgow's Minimum Essential Medium without glutamine. It was supplemented with nucleosides, Glutamic acid, asparagine, and 10% fetal bovine serum.

The cells were transfected with the pBC40 construct using FuGENE 6® (Roche Molecular Biochemicals) using 3 μl of FuGENE 6 per 1 μg plasmid DNA. Transfected cells containing the GAA plasmid were selected with increasing concentrations of methionine sulfoxamine to obtain a stable expressing cell line. One example of such a cell line expressing acid α glucosidase is clone number 3.49.13.1.

Preparation of Ricin resistant GAA expressing CHO cell A confluent T-150 flask of clone number 3.49.13.1 was trypsinized and counted. Cells were then washed with Dulbeco's Phosphate Buffered Saline (DPBS) and resuspended to 1×10⁶ cells/ml in a total volume of 10 ml of serum-free GMEM containing Ricinus communis-II lectin (RCA-II; EY Laboratories) at a concentration of 0.13 mg/ml. Cells were incubated at 37° C. for one hour. Next, the cell suspension was brought to a final volume of 415 ml with GMEM containing 10% dialyzed FBS. Cells were then plated out into 20×96 well plates at 5000 cells/ well. Cells were cultured until colony formation was evident. Ten ricin-resistant clones numbered R3.1-R3.10 were cloned by limiting dilution and banked in liquid nitrogen.

To demonstrate whether the ricin-resistant clones actually produced glycoproteins containing no complex type oligosaccharides, cell cultures were grown in the presence of ³⁵S methionine containing media. After 16 hours ³⁵S labeled GAA was purified from the media by immunoprecipitation using specific polyclonal antiserum and the molecular weights compared by SDS-PAGE autoradiography before and after incubation with endoglycosidase H and glycopeptidase F. Endoglycosidase H cleaves only high mannose or hybrid type N-glycans whereas glycopetidase F hydrolyzes all types of N-glycan chains except for those containing α1,3-bound core fucose residues. Clones expressing GAA with the same molecular weights after incubation with endoglycosidase H and glycopeptidase F were considered as not expressing complex type N-glycans and were subjected to further analysis. All ten ricin-resistant clones showed identical banding patterns by SDS-PAGE suggesting all ten clones contained only high mannose oligosaccharide side chains on the expressed GAA. However, additional assays looking at Gnt-1 activity showed that R3.6 and R3.9 were not Lec 1's even though they were ricin resistant. Further analyses including cell growth rates, GAA production levels and GAA phosphorylation by GlcNAc-phosphotransferase was carried out. Among the ten clones, R3.3 was selected as the best cell line for producing GAA with the necessary N-glycan structures for in vitro phosphorylation using GlNac phosphotransferase and phosphodiester α-GlcNAcase.

Phosphorylation Efficiency of rh-GAA from Cultures Containing Mannosidase Inhibitors

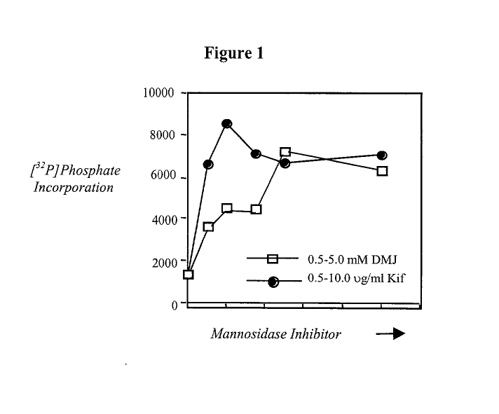

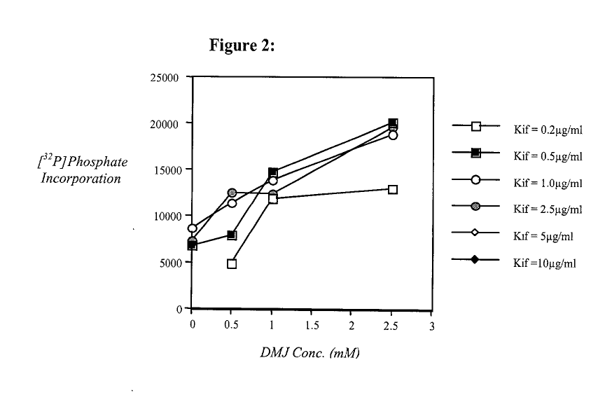

To determine whether mannosidase inhibitors can increase phosphorylation efficiency of recombinant human acid alpha glucosidase (rh-GAA), a CHO cell line (GAA LEC clone R3.3) expressing rh-GAA was grown in various conditions that contained different amounts of the following mannosidase the inhibitors: Kifunensin; Deoxymannojirimycin (DMJ); or combinations of both inhibitors. Conditioned media from each inhibitor condition containing 6 μg of GAA (based on GAA activity assay) was then incubated with purified bovine GlcNAc phosphotransferase (Pt'ase) and [³²P]UDP-GlcNAc. Subsequently, each phosphorylation reaction was loaded onto a concanavalin A-sepharose column to capture glycoproteins, e.g., rh-GAA. The concanavalin A-sepharose was washed and the resin was counted in a liquid scintillation counter to measure the ³²P incorporation, i.e., the extent of phosphorylation on rh-GAA. The results are summarized in Table 1 below and FIGS. 1 and 2.

TABLE 1
Incorporation of [³²P] Phosphate on rh-GAA

| | Concentration of | [³²P] Phosphate |
|---|---|---|
| Sample | Mannosidase Inhibitor | Incorporation (cpm) |
| No inhibitor | 0 | 462 |
| DMJ only | 0.5 mM | 3656 |
| DMJ only | 1.0 mM | 4500 |
| DMJ only | 1.75 mM | 4450 |
| DMJ only | 2.5 mM | 7258 |
| DMJ only | 5.0 mM | 6413 |
| Kifunensin only | 0.5 μg/ml | 6675 |
| Kifunensin only | 1.0 μg/ml | 8585 |
| Kifunensin only | 2.5 μg/ml | 7147 |
| Kifunensin only | 5.0 μg/ml | 6717 |
| Kifunensin only | 10.0 μg/ml | 7116 |
| DMJ + Kifunensine | 0.5 mM DMJ/ | 4866 |
| | 0.2 μg/ml Kifunensine | |
| DMJ + Kifunensine | 0.5 mM DMJ/ | 7806 |
| | 0.5 μg/ml Kifunensine | |
| DMJ + Kifunensine | 0.5 DMJ/ | 11296 |
| | 1.0 μg/ml Kifunensine | |
| DMJ + Kifunensine | 0.5 mM DMJ/ | 12417 |
| | 2.5 μg/ml Kifunensine | |
| DMJ + Kifunensine | 1.0 mM DMJ/ | 11821 |
| | 0.2 μg/ml Kifunensine | |
| DMJ + Kifunensine | 1.0 mM DMJ/ | 14760 |
| | 0.5 μg/ml Kifunensine | |
| DMJ + Kifunensine | 1.0 mM DMJ/ | 13875 |
| | 1.0 μg/ml Kifunensine | |
| DMJ + Kifunensine | 1.0 mM DMJ/ | 12305 |
| | 2.5 μg/ml Kifunensine | |
| DMJ + Kifunensine | 2.5 mM DMJ/ | 14250 |
| | 0.2 μg/ml Kifunensine | |
| DMJ + Kifunensine | 2.5 mM DMJ/ | 20024 |
| | 0.5 μg/ml Kifunensine | |
| DMJ + Kifunensine | 2.5 mM DMJ/ | 18865 |
| | 1.0 μg/ml Kifunensine | |
| DMJ + Kifunensine | 2.5 mM DMJ/ | 12305 |
| | 2.5 μg/ml Kifunensine | |

Table 1 summarizes how the use of the mannosidase inhibitors DMJ and Kifunensin profoundly affected the phosphorylation efficiency of rh-GAA from conditioned media. GAA that was cultured without mannosidase inhibitors showed very low levels of [³²P]phosphate incorporation, i.e., GlcNAc-phosphotrasnsferase-dependent phosphorylation. In contrast, increasing amounts of either DMJ or Kifunensin alone was enough to greatly enhance the phosphorylation reaction (FIG. 1). In addition, the combination of these two inhibitors increased the phosphorylation of GAA nearly 3-fold compared to GAA that was cultured in either DMJ or Kifunensin alone (FIG. 2). These mannosidase inhibitors prevented the trimming of the carbohydrate structures on GAA and allowed these N-glycans to remain as high mannose chains. As a result, these high mannose N-glycans are better substrates for phosphotransferase.

Obviously, numerous modifications and variations on the present invention are possible in light of the above teachings. It is therefore to be understood that within the scope of the appended claims, the invention may be practiced otherwise than as specifically described herein.

What is claimed is:

1. A method of producing a high mannose glycoprotein comprising
a. introducing and expressing a polynucleotide encoding a glycoprotein into a mammalian cell;
b. culturing the mammalian cell in the presence of a lectin in an amount sufficient to obtain a lectin resistant mammalian cell;
c. isolating the lectin resistant mammalian cell;
d. culturing said lectin resistant mammalian cell in the presence of deoxymannojirimycin and kifunensine in an amount and for a time to inhibit glycosylation of the glycoprotein; and
e. collecting the high mannose glycoprotein.

2. The method of claim 1, wherein said lectin is selected from the group consisting of ricin, concanavalin A, erthroglutinin, lymphoagglutanin, and wheat germ agglutinin.

3. The method of claim 2, wherein said lectin is ricin.

4. The method of claim 1, wherein said glycoprotein is a lysosomal hydrolase.

5. The method of claim 4, wherein said lysosomal hydrolase is selected from the group consisting of α-glucosidase, α-L-iduronidase, α-galactosidase A, arylsulfatase, N-acetylgalactosamine-6-sulfatase or β-galactosidase, iduronate 2-sulfatase, ceramidase, galactocerebrosidase, β-glucuronidase, Heparan N-sulfatase, N-Acetyl-α-glucosaminidase, Acetyl CoA-α-glucosaminide N-acetyl transferase, N-acetyl-glucosamine-6sulfatase, Galactose 6-sulfatase, Arylsulfatase A, Arylsulfatase B, Arylsulfatase C, Arylsulfatase A Cerebroside, Ganglioside, Acid β-galactosidase G_{M1} Galglioside, Acid β-galactosidase, Hexosaminidase A, Hexosaminidase B, α-fucosidase, α-N-Acetyl galactosaminidase, Glycoprotein Neuraminidase, Aspartylglucosamine amidase, Acid Lipase, Acid Ceramidase, Lysosomal Sphingomyelinase and Sphingomyelinase.

6. The method of claim 5, wherein said lysosomal hydrolase is acid α-glucosidase.

7. The method of claim 1, further comprising contacting the collected glycoprotein with a GlcNAc-phosphotransferase.

8. The method of claim 7, wherein the GlcNAc-phosphotransferase comprises SEQ ID NO:2.

9. The method of claim 7, wherein the GlcNAc-phosphotransferase comprises SEQ ID NO:2 and SEQ ID NO:7.

10. The method of claim 7, wherein the GlcNAc-phosphotransferase comprises SEQ ID NOS:4, 5 and 7.

11. The method of claim 7, wherein the GlcNAc-phosphotransferase is encoded by a nucleotide sequence comprising SEQ ID NO:1 or a nucleotide sequence that hybridizes under stringent conditions to the complement of SEQ ID NO:1.

12. The method of claim 7, wherein the GlcNAc-phosphotransferase comprises an α-subunit and a β subunit, which are encoded by a nucleotide sequence comprising SEQ ID NO:3 or a nucleotide sequence that hybridizes under stringent conditions to the complement of SEQ ID NO:3; and a γ subunit, which is encoded by a nucleotide sequence comprising SEQ ID NO:6 or a nucleotide sequence that hybridizes under stringent conditions to the complement of SEQ ID NO:6.

13. The method of claim 7, further comprising purifying said glycoprotein after said contacting.

14. The method of claim 7, wherein after said contacting with GlcNAc-phosphotransferase the method further comprises contacting with said glycoprotein with a phosphodiester α-GlcNAcase.

15. The method of claim 14, wherein said phosphodiester α-GlcNAcase comprises an amino acid sequence of SEQ ID NO:18.

16. The method of claim 14, wherein said phosphodiester α-GlcNAcase is encoded by a nucleotide sequence comprising SEQ ID NO:17 or a nucleotide sequence that hybridizes under stringent conditions to the complement of SEQ ID NO:17.

17. The method of claim 14, further comprising purifying said glycoprotein after said contacting.

18. The method of claim 1, wherein said deoxymannojirimycin is present in an amount from about 0.1 mM to about 5.0 mM.

19. The method of claim 1, wherein said kifunensine is in present in an amount from about 0.1 μg/ml to about 10 μg/ml.

20. A high mannose glycoprotein produced by the method of claim 1.

21. A method of producing a high mannose glycoprotein comprising
a. culturing a lectin resistant mammalian cell in the presence of deoxymannojirimycin and kifunensine in an amount and for a time to inhibit glycosylation of the glycoprotein; and
b. collecting the high mannose glycoprotein.

22. The method of claim 21, wherein said lectin is selected from the group consisting of ricin, concanavalin A, erthroglutinin, lymphoagglutanin, and wheat germ agglutinin.

23. The method of claim 22, wherein said lectin is ricin.

24. The method of claim 21, wherein said glycoprotein is a lysosomal hydrolase.

25. The method of claim 24, wherein said lysosomal hydrolase is selected from the group consisting of α-glucosidase, α-L-iduronidase, α-galactosidase A, arylsulfatase, N-acetylgalactosamine-6-sulfatase or β-galactosidase, iduronate 2-sulfatase, ceramidase, galactocerebrosidase, β-glucuronidase, Heparan N-sulfatase, N-Acetyl-α-glucosaminidase, Acetyl CoA-α-glucosaminide N-acetyl transferase, N-acetyl-glucosamine-6sulfatase, Galactose 6-sulfatase, Arylsulfatase A, Arylsulfatase B, Arylsulfatase C, Arylsulfatase A Cerebroside, Ganglioside, Acid β-galactosidase G_{M1} Galglioside, Acid β-galactosidase, Hexosaminidase A, Hexosaminidase B, α-fucosidase, α-N-Acetyl galactosaminidase, Glycoprotein Neuraminidase, Aspartylglucosamine amidase, Acid Lipase, Acid Ceramidase, Lysosomal Sphingomyelinase and Sphingomyelinase.

26. The method of claim 25, wherein said lysosomal hydrolase is acid α-glucosidase.

27. The method of claim 21, further comprising contacting the collected glycoprotein with a GlcNAc-phosphotransferase.

28. The method of claim 27, wherein the GlcNAc-phosphotransferase comprises SEQ ID NO:2.

29. The method of claim 27, wherein the GlcNAc-phosphotransferase comprises SEQ ID NO:2 and SEQ ID NO:7.

30. The method of claim 27, wherein the GlcNAc-phosphotransferase comprises SEQ ID NOS:4, 5 and 7.

31. The method of claim 27, wherein the GlcNAc-phosphotransferase is encoded by a nucleotide sequence comprising SEQ ID NO:1 or a nucleotide sequence that hybridizes under stringent conditions to the complement of SEQ ID NO:1.

32. The method of claim 27, wherein the GlcNAc-phosphotransferase comprises an α-subunit and a β subunit, which are encoded by a nucleotide sequence comprising SEQ ID NO:3 or a nucleotide sequence that hybridizes under stringent conditions to the complement of SEQ ID NO:3; and a γ subunit, which is encoded by a nucleotide sequence comprising SEQ ID NO:6 or a nucleotide sequence that hybridizes under stringent conditions to the complement of SEQ ID NO:6.

33. The method of claim 27, further comprising purifying said glycoprotein after said contacting.

34. The method of claim 27, wherein after said contacting with GlcNAc-phosphotransferase the method further comprises contacting with said glycoprotein with a phosphodiester α-GlcNAcase.

35. The method of claim 34, wherein said phosphodiester α-GlcNAcase comprises an amino acid sequence of SEQ ID NO:18.

36. The method of claim 34, wherein said phosphodiester α-GlcNAcase is encoded by a nucleotide sequence comprising SEQ ID NO:17 or a nucleotide sequence that hybridizes under stringent conditions to the complement of SEQ ID NO:17.

37. The method of claim 34, further comprising purifying said glycoprotein after said contacting.

38. The method of claim 21, wherein said deoxymannojirimycin is present in an amount from about 0.1 mM to about 5.0 mM.

39. The method of claim 21, wherein said kifunensine is in present in an amount from about 0.1 μg/ml to about 10 μg/ml.

40. A high mannose glycoprotein produced by the method of claim 1.

41. A method of treating a patient suffering from a lysosomal storage disease comprising administering to said patient a lysosomal hydrolase in an amount sufficient to treat said disease, wherein said lysosomal hydrolase is obtained by a method comprising:
a. culturing a lectin resistant mammalian cell in the presence of deoxymannojirimycin and kifunensine in an amount and for a time to inhibit glycosylation of the glycoprotein;
b. collecting the high mannose glycoprotein;
c. collecting the lysosomal hydrolase from said lectin resistant cells;
d. contacting the collected lysosomal hydrolase with a GlcNAc-phosphotransferase; and
e. contacting said lysosomal hydrolase with a phosphodiester α GlcNACase after said contacting with a GlcNAc-phosphotransferase.

42. The method of claim 41, wherein said lectin is selected from the group consisting of ricin, concanavalin A, erthroglutinin, lymphoagglutanin, and wheat germ agglutinin.

43. The method of claim 42, wherein said lectin is ricin.

44. The method of claim 41, wherein said glycoprotein is a lysosomal hydrolase.

45. The method of claim 44, wherein said lysosomal hydrolase is selected from the group consisting of α-glucosidase, α-L-iduronidase, α-galactosidase A, arylsulfatase, N-acetylgalactosamine-6-sulfatase or β-galactosidase, iduronate 2-sulfatase, ceramidase, galactocerebrosidase, β-glucuronidase, Heparan N-sulfatase, N-Acetyl-α-glucosaminidase, Acetyl CoA-α-glucosaminide N-acetyl transferase, N-acetyl-glucosamine-6sulfatase, Galactose 6-sulfatase, Arylsulfatase A, Arylsulfatase B, Arylsulfatase C, Arylsulfatase A Cerebroside, Ganglioside, Acid β-galactosidase G_{M1} Galglioside, Acid β-galactosidase, Hexosaminidase A, Hexosaminidase B, α-fucosidase, α-N-Acetyl galactosaminidase, Glycoprotein Neuraminidase, Aspartylglucosamine amidase, Acid Lipase, Acid Ceramidase, Lysosomal Sphingomyelinase and Sphingomyelinase.

46. The method of claim 45, wherein said lysosomal hydrolase is acid α-glucosidase.

47. The method of claim 45, wherein the GlcNAc-phosphotransferase comprises SEQ ID NO:2.

48. The method of claim 45, wherein the GlcNAc-phosphotransferase comprises SEQ ID NO:2 and SEQ ID NO:7.

49. The method of claim 45, wherein the GlcNAc-phosphotransferase comprises SEQ ID NOS:4, 5 and 7.

50. The method of claim 45, wherein the GlcNAc-phosphotransferase is encoded by a nucleotide sequence comprising SEQ ID NO:1 or a nucleotide sequence that hybridizes under stringent conditions to the complement of SEQ ID NO:1.

51. The method of claim 45, wherein the GlcNAc-phosphotransferase comprises an α-subunit and a β subunit, which are encoded by a nucleotide sequence comprising SEQ ID NO:3 or a nucleotide sequence that hybridizes under stringent conditions to the complement of SEQ ID NO:3; and a γ subunit, which is encoded by a nucleotide sequence comprising SEQ ID NO:6 or a nucleotide sequence that hybridizes under stringent conditions to the complement of SEQ ID NO:6.

52. The method of claim 45, wherein said phosphodiester α-GlcNAcase comprises an amino acid sequence of SEQ ID NO:18.

53. The method of claim 45, wherein said phosphodiester α-GlcNAcase is encoded by a nucleotide sequence comprising SEQ ID NO:17 or a nucleotide sequence that hybridizes under stringent conditions to the complement of SEQ ID NO:17.

54. The method of claim 45, wherein said deoxymannojirimycin is present in an amount from about 0.1 mM to about 5.0 mM.

55. The method of claim 45, wherein said kifunensine is in present in an amount from about 0.1 μg/ml to about 10 μg/ml.

56. A method of producing a high mannose glycoprotein comprising
a. a step culturing mammalian cells expressing said high mannose glycoprotein under conditions to produce the high mannose glycoprotein; and
b. a step for collecting the glycoprotein.

57. The method of claim 56, wherein said lectin is selected from the group consisting of ricin, concanavalin A, erthroglutinin, lymphoagglutanin, and wheat germ agglutinin.

58. The method of claim 57, wherein said lectin is ricin.

59. The method of claim 56, wherein said glycoprotein is a lysosomal hydrolase.

60. The method of claim 59 wherein said lysosomal hydrolase is selected from the group consisting of α-glucosidase, α-L-iduronidase, α-galactosidase A, arylsulfatase, N-acetylgalactosamine-6-sulfatase or, β-galactosidase, iduronate 2-sulfatase, ceramidase, galactocerebrosidase, β-glucuronidase, Heparan N-sulfatase, N-Acetyl-α-glucosaminidase, Acetyl CoA-α-glucosaminide N-acetyl transferase, N-acetyl-glucosamine-6sulfatase, Galactose 6-sulfatase, Arylsulfatase A, Arylsulfatase B, Arylsulfatase C, Arylsulfatase A Cerebroside, Ganglioside, Acid β-galactosidase G_{M1} Galglioside, Acid β-galactosidase, Hexosaminidase A, Hexosaminidase B, α-fucosidase, α-N-Acetyl galactosaminidase, Glycoprotein Neuraminidase, Aspartylglucosamine amidase, Acid Lipase, Acid Ceramidase, Lysosomal Sphingomyelinase and Sphingomyelinase.

61. The method of claim 60, wherein said lysosomal hydrolase is acid α-glucosidase.

62. The method of claim 56, further comprising a step for transferring a N-acetylglucosamine-1-phosphate from UDP-GlcNAc to said glycoprotein.

63. The method of claim 62, further comprising a step for purifying said glycoprotein comprising a N-acetylglucosamine-1-phosphate.

64. The method of claim 62, further comprising a step for removing an N-acetylglucosamine from said glycoprotein.

65. A high mannose glycoprotein produced by the method of claim 56.
